SEQUENCE LISTING (26 sequences)

SEQ ID NO: 1 - DNA, 19 bp, Artificial Sequenc
  Synthetic
acaagcuggucagcuucua

SEQ ID NO: 2 - DNA, 19 bp, Artificial Sequenc
  Synthetic
uagaagcugaccagcuugu

SEQ ID NO: 3 - DNA, 19 bp, Artificial Sequenc
  Synthetic
gacaagcuggucagcaaga

SEQ ID NO: 4 - DNA, 21 bp, Artificial Sequenc
  Synthetic
  misc_feature (20)..(21): n is a, c, g, t or u
agaagcugaccagcuugucnn

SEQ ID NO: 5 - DNA, 20 bp, Artificial Sequenc
  Synthetic
  misc_feature (20)..(20): n is a, c, g, t or u
gacaagcuggucagcuucun

SEQ ID NO: 6 - DNA, 20 bp, Artificial Sequenc
  Synthetic
  misc_feature (20)..(20): n is a, c, g, t or u
agaagcugaccagcuugucn

SEQ ID NO: 7 - DNA, 19 bp, Artificial Sequenc
  Synthetic
gacaagcuggucagcuucu

SEQ ID NO: 8 - DNA, 19 bp, Artificial Sequenc
  Synthetic
agaagcugaccagcuuguc

SEQ ID NO: 9 - DNA, 19 bp, Artificial Sequenc
  Synthetic
acaagcuggucagcuagau

SEQ ID NO: 10 - DNA, 21 bp, Artificial Sequenc
  Synthetic
  misc_feature (20)..(21): n is a, c, g, t or u
uagaagcugaccagcuugunn

SEQ ID NO: 11 - DNA, 19 bp, Artificial Sequenc
  Synthetic
gacaagcuggucagcuuca

SEQ ID NO: 12 - DNA, 21 bp, Artificial Sequenc
  Synthetic
  misc_feature (20)..(21): n is a, c, g, t or u
agaagcugaccagcuugucnn

SEQ ID NO: 13 - DNA, 19 bp, Artificial Sequenc
  Synthetic
cucaccgacaagcugguca

SEQ ID NO: 14 - DNA, 21 bp, Artificial Sequenc
  Synthetic
ugaccagcuugucggugaggu

SEQ ID NO: 15 - DNA, 19 bp, Artificial Sequenc
  Synthetic
gacaagcuggucagcuucu

SEQ ID NO: 16 - DNA, 21 bp, Artificial Sequenc
  Synthetic
agaagcugaccagcuugucgg

SEQ ID NO: 17 - DNA, 19 bp, Artificial Sequenc
  Synthetic
ucaccgacaagcuggucag

SEQ ID NO: 18 - DNA, 21 bp, Artificial Sequenc
  Synthetic
cugaccagcuugucggugagg

SEQ ID NO: 19 - DNA, 19 bp, Artificial Sequenc
  Synthetic
caccgacaagcuggucagc

SEQ ID NO: 20 - DNA, 21 bp, Artificial Sequenc
  Synthetic
gcugaccagcuugucggugag

SEQ ID NO: 21 - DNA, 19 bp, Artificial Sequenc
  Synthetic
accgacaagcuggucagcu

SEQ ID NO: 22 - DNA, 21 bp, Artificial Sequenc
  Synthetic
agcugaccagcuugucgguga

SEQ ID NO: 23 - DNA, 19 bp, Artificial Sequenc
  Synthetic
ccgacaagcuggucagcuu

SEQ ID NO: 24 - DNA, 21 bp, Artificial Sequenc
  Synthetic
aagcugaccagcuugucggug

SEQ ID NO: 25 - DNA, 19 bp, Artificial Sequenc
  Synthetic
cgacaagcuggucagcuuc

SEQ ID NO: 26 - DNA, 21 bp, Artificial Sequenc
  Synthetic
gaagcugaccagcuugucggu

(19) United States
(12) Patent Application Publication
(10) Pub. No.: US 2023/0416751 A1
(43) Pub. Date: Dec. 28, 2023

(54) MATERIALS AND METHODS FOR TREATING CORNEAL DYSFUNCTION

(71) Applicant: UNIVERSITY OF MIAMI, Miami, FL (US)

(72) Inventors: Alfonso L Sabater Gozalvo, Miami, FL (US); Juan Pablo De Rivero Vaccari, Miami, FL (US); Robert W. Keane, Miami, FL (US); Enrique L. Salero-Coca, Aventura, FL (US)

(21) Appl. No.: 18/036,494

(22) PCT Filed: Nov. 12, 2021
(86) PCT No.: PCT/US21/59197

§ 371 Date: May 11, 2023

Related U.S. Application Data
(60) Provisional application No. 63/113,736, filed on Nov. 13, 2020.

(51) Int. Cl. C12N 15/113; C07K 16/40; C07K 16/28; A61P 27/02

(52) U.S. Cl. CPC C12N 15/1137; C12N 15/1138; C07K 16/40; C07K 16/28; A61P 27/02; C12N 2310/14

(57) ABSTRACT

The present disclosure relates to materials and methods for treating or preventing corneal dysfunction or ocular surface disease.

### CROSS REFERENCE TO RELATED APPLICATIONS

This application claims priority to U.S. Provisional Patent Application No. 63/113,736, filed on Nov. 13, 2020, which is incorporated by reference in its entirety.

### FIELD OF THE INVENTION

The present disclosure relates to materials and methods for treating and/or preventing corneal dysfunction or ocular surface diseases.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “56227A_Seqlisting.txt”, which was created on Nov. 11, 2021 and is 5,346 bytes in size. The subject matter of the Sequence Listing is incorporated herein in its entirety by reference.

### BACKGROUND

The endothelium is the innermost layer in the human cornea, acting as a barrier with pump functions that are critical to maintaining corneal transparency or deturgescence^{1,2}. In contrast to the epithelium, which has a self-renewing capacity, the endothelium does not have the capacity to regenerate^{3,4}. Thus, in conditions such as Fuchs' endothelial dystrophy, Pseudophakic bullous keratopathy (PB K)^{5,6} or corneal graft failure^{7,8}, where the corneal endothelium is affected and cell density may be compromised, the nonregenerative capacity of the human corneal endothelium translates into potentially deleterious outcomes. To date, there is no preventive therapy available for any endothelial corneal degeneration, and allogenic corneal transplantation is the main option to treat these conditions⁹. More than 40,000 corneal transplants are performed each year in the United States, and over 50% of them were done because of corneal endothelial dysfunction.

Triggering factors involved in the previously mentioned conditions have been an object of interest to address the causes and find new treatments. Some intraocular mechanisms have been associated with the chronic loss of CEC in patients with corneal endothelial dysfunction. An elevation of intraocular proinflammatory cytokines such as IFN-γ and TNF-α have been found in patients with PBK and low endothelial cell density^{10,11}. No significant differences in intraocular proinflammatory cytokine levels have been found between patients using topical steroids and those who are not steroid users¹¹, suggesting that an alternative pathway may be contributing to a form of subclinical inflammation that underlies these conditions.

Ocular surface damage, which manifests as punctate epithelial erosions in the conjunctiva and/or cornea, can be the end result of many disease processes including dry eye disease (DED), medication use, and environmental exposure (Bron et al., Ocul. Surf., 15:438-510, 2017; Mantelli et al., Curr. Opin. Allergy Clin. Immunol., 11:464-470, 2011). Ocular surface damage can also be caused by a number of systemic (Askeroglu et al., Plast. Reconstr. Surg., 131:159-167, 2013) and topical medications (Rosin et al., Clin. Ophthalmol., 7:2131-2135, 2013; Baudoin et al., Ophthalmology, 106:556-563, 1999). In particular, topical ocular hypotensive medications have been associated with ocular surface damage, both due their preservatives and active agents (Anwar et al., Curr. Opin. Ophthalmol., 24:136-143, 1999; Sedlak et al., BMC Ophthalmol., 21:319, 2021).

### SUMMARY

The disclosure provides a method of treating or preventing corneal dysfunction in a mammalian subject (e.g., human) in need thereof. In one embodiment, the method comprises administering to a subject a composition that comprises an inflammasome inhibitor in an amount effective to treat or prevent the corneal dysfunction. The disclosure also provides a method of treating or preventing ocular surface disease (or ocular surface damage) in a mammalian subject (e.g., human) in need thereof. In another embodiment, the method comprises administering to a subject a composition that comprises an inflammasome inhibitor in an amount effective to treat or prevent ocular surface disease. In various aspects, the inflammasome inhibitor inhibits caspase-1, adaptor protein apoptosis-associated speck-like protein containing a caspase recruitment domain (ASC), nucleotide-binding domain leucine-rich repeat pyrin domain containing 1 (NLRP1), nucleotide-binding domain leucine-rich repeat pyrin domain containing 2 (NLRP2), nucleotide-binding domain leucine-rich repeat pyrin domain containing 3 (NLRP3), or absent in melanoma 2 (AIM2). In various aspects, the inhibitor is an antibody, an antisense oligonucleotide, or siRNA.

In various aspects, the corneal dysfunction is corneal endothelial dysfunction, acute or chronic corneal endothelial cell (CEC) loss, bullous keratopathy (PBK), Fuchs' endothelial dystrophy, corneal transplant failure, corneal transplant rejection, corneal inflammation, corneal edema, corneal degeneration, corneal melting, or corneal ectasia.

In various aspects, the ocular surface disease is dry eye syndrome, meibomian gland dysfunction, blepharitis, ocular rosacea allergic conjunctivitis (including seasonal allergic conjunctivitis (SAC), perennial allergic conjunctivitis (PAC), atopic keratoconjunctivitis (AKC), vernal keratoconjunctivitis (VKC), and giant papillary conjunctivitis (GPC)), chemical and thermal ocular burns, Pterygium, Pinguecula, conjunctivochalasis or limbal stem cell deficiency.

In another aspect, described herein is a method of diagnosing ocular surface damage in a mammalian subject in need thereof, the method comprising: determining a level of caspase-1 in a tear sample from the subject, wherein an elevated level of caspase-1 in the sample compared to a control sample identifies the subject as likely to be suffering from ocular surface damage; and administering an inflammasome inhibitor to the subject in an amount effective to treat the ocular surface damage.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “one embodiment,” “some embodiments,” “various embodiments,” “related embodiments,” each of these types of embodiments is a non-limiting example of a feature that is intended to be combined with any other feature, or combination of features, described herein without having to list every possible combination. Such features or combinations of features apply to any of the aspects of the invention.

The headings herein are for the convenience of the reader and not intended to be limiting. Additional aspects, embodiments, and variations of the invention will be apparent from the Detailed Description and/or drawings and/or claims.

### BRIEF DESCRIPTION OF THE DRAWINGS

FIGS. 1A-1B show the role of inflammation in corneal endothelial cell inflammasome activation. The graphs illustrate caspase-1 (FIG. 1A) and IL-1β (FIG. 1B) expression in human CEC exposed to proinflammatory cytokines, TNF-α (100 ng/ml), IFN-γ (100 ng/ml) and caspase-1 inhibitor, Ac-YVAD-cmk (30 ng/ml) for 48 hours. Data presented as mean+/−SEM. N=4 per group. *p<0.05, **p<0.001, ****p<0.0001, ns=not significant.

FIG. 2 shows inflammation-induced endothelial cell death protection by caspase-1 inhibition. Lactate dehydrogenase (LDH) release assay in human CEC exposed to proinflammatory cytokines TNF-α (100 ng/ml), IFN-γ (100 ng/ml) and caspase-1 inhibitor, Ac-YVAD-cmk (30 ng/ml) for 48 hours. Data presented as mean+/−SEM. N=3 per group. *p<0.05.

FIG. 3 shows the role of oxidative stress in corneal endothelial cell pyroptosis. Reactive oxygen species (ROS) generation detection assay in human CEC exposed to proinflammatory cytokines TNF-α (100 ng/ml), IFN-γ (100 ng/ml) and caspase-1 inhibitor, Ac-YVAD-cmk (30 ng/ml) for 48 hours. Data presented as mean+/−SEM. N=3 per group. *p<0.05, **p<0.001.

FIG. 4 shows ASC in the aqueous humor of patients with PBK. ASC detection in the aqueous humor of patients with PBK. Data presented as mean+/−SEM. N=5 (PBK) and 8 (control). ****p<0.0001. Since ASC levels were undetected in the control samples, the lower limit of quantitation (13.1 pg/mL) was assumed in these groups. Controls were healthy patients. Aqueous humor was obtained at the beginning of the surgery in all cases.

FIG. 5 shows IL-18 in the aqueous humor of patients with PBK. IL-18 detection in the aqueous humor of patients with PBK. Data presented as mean+/−SEM. N=4 (PBK) and 8 (control). *p 0.0275. Since IL-18 levels were undetected in the control samples, the lower limit of quantitation (0.390 pg/mL) was assumed in this group. Controls were healthy patients undergoing standard phacoemulsification cataract surgery. PBK were patients undergoing corneal transplantation. Aqueous humor was obtained right at the beginning of the surgery in all cases.

FIG. 6 shows ASC in the aqueous humor of patients with PBK and Fuchs' dystrophy. Data presented as mean+/−SEM. N=5 (PBK), 4 (Fuchs') and 8 (control). ****p<0.0001. Since ASC levels were undetected in the control and Fuchs' samples, the lower limit of quantitation (13.1 pg/mL) was assumed in these group. Controls were healthy patients undergoing standard phacoemulsification cataract surgery. Fuchs' and PBK were patients undergoing corneal transplantation. Aqueous humor was obtained right at the beginning of the surgery in all cases (UM IRB 20190334).

FIG. 7 shows ASC in the aqueous humor of patients with PBK and failed corneal transplants. Data presented as mean+/−SEM. N=5 (PBK), 3 (failed transplants) and 8 (control). ****p<0.0001. Since ASC levels were undetected in the control samples, the lower limit of quantitation (13.1 pg/mL) was assumed in these group. Controls were healthy patients. PBK were patients undergoing corneal transplantation. Aqueous humor was obtained right at the beginning of the surgery in all cases (UM IRB 20190334).

FIGS. 8A-8F are graphs showing the clinical presentation of the ocular surface in patients with dry eye disease (DED), glaucoma (GLC), and healthy controls (C). FIG. 8A: Patients using glaucoma medications presented with the highest CS scores (NEI scale), followed by the DED group, that were statistically significant when compared to those of the controls. N=C: 19, DED: 61, GLC: 32. FIG. 8B: The conjunctival staining scores (NEI scale) were significantly higher in patients using antiglaucoma medications when compared to the controls. N=C: 19, DED: 49, GLC: 32. FIG. 8C: Patients using antiglaucoma medications showed the highest redness scores (Effron scale) when compared to the other two groups. N=C: 5, DED: 16, GLC: 32. FIG. 8D: Controls had the highest Schirmer's test results (mm) when compared to the other two groups. N=C: 20, DED: 61, GLC: 32. FIG. 8E: Tear break-up time (TBUT) (Sec) were longer in controls when compared to the group using glaucoma medications. N=C: 17, DED: 59, GLC: 32. FIG. 8F: The scores of OSDI questionnaires were significantly higher in the glaucoma and DED group, compared to the controls N=C: 20, DED, 61, GLC: 32. Statistical significance was derived from linear mixed models. Data presented as boxplots with the 5th and 95th percentile.

FIG. 9 is a graph showing caspase-1 in the tears of patients with dry eye disease (DED), glaucoma (GLC) and healthy controls (C). The population using glaucoma medications presented the highest caspase-1 concentration levels in their tears. Statistical significance was derived from linear mixed models. N=113; C: 20, DED: 61, GLC: 32. Data presented as boxplots with the 5th and 95th percentile.

FIG. 10 is a graph showing the Receiver Operating Characteristics (ROC) of caspase-1 for the diagnosis of ocular surface damage. AUC of caspase-1 in patients diagnosed with ocular surface damage as determined by whether patients presented corneal staining (>3 points on NEI scale) or not (<3 points on NEI scale). Using 82.85 pg/mL as the cut-off point, the AUC for caspase-1 is 0.7, with a sensitivity of 73% and specificity of 64%.

FIGS. 11A-11C are graphs showing the inflammasome activation in aqueous humor of patients with corneal graft failure. Expression of ASC (FIG. 11A), IL-18 (FIG. 11B) and IL-1β (FIG. 11C) in AqH of patients with CGF. N. 12=Healthy eyes; N. 7=CGF. Data presented as mean±SEM. *p<0.0246, **p<0.0063, ****p<0.0001. The Lower Limit of Quantitation of ASC, IL-18 and IL-1β was assumed in samples with undetected levels of these proteins (13.00 pg/mL, 0.39 pg/mL, and 0.20 pg/mL respectively).

FIG. 12 is a graph showing inflammasone activation in aqueous humor of patients with PBK and Fuchs' dystrophy. Expression of ASC in AqH of patients with corneal endothelial dysfunction. N. 12=Healthy eyes; N. 4=Fuchs' dystrophy; N. 5=PBK. Data presented as mean±SEM. ****p<0.0001, ns=no significance. The Lower Limit of Quantitation of ASC was assumed in samples with undetected levels of these proteins (13.00 pg/mL).

FIG. 13 is a graph showing inflammasome activation in corneal endothelial cells after aqueous humor exposure. Caspase-1 expression in HCEC-B4G12 endothelial cells after 60 minutes of aqueous humor (AqH) exposure from patients with CGF and from healthy patients. N. 3=Failed corneal transplants; N. 3=Healthy eyes; N. 3=No AqH exposure.

FIG. 14 is a graph showing the levels of caspase-1 levels in HCEC-B4G12 (Human Corneal Endothelial Cell Line) cells exposed to ASC siRNA or YVAD compared to positive control cells treated only with TNFα and INFγ.

### DETAILED DESCRIPTION

Corneal endothelial dysfunction is a primary cause of corneal transplant failure, even in eyes without evidence of immunologic rejection. The corneal endothelium is responsible for regulating corneal stromal hydration and transparency. Endothelial cell loss has been associated with an elevation of proinflammatory cytokines such as interferon (IFN-γ) and tumor necrosis factor (TNF) in the aqueous humor. The exact mechanisms responsible for endothelial cell loss are poorly understood, and there is no treatment available for this condition.

The disclosure provides materials and methods for treating or preventing corneal dysfunction or ocular surface disease. In one aspect, the disclosure provides a method of treating or preventing corneal dysfunction in a mammalian subject. In another aspect, the disclosure provides a method of treating or preventing ocular surface disease in a mammalian subject. The method comprises administering to the subject an inflammasome inhibitor in an amount effective to treat the corneal dysfunction, prevent the corneal dysfunction, treat the ocular surface disease, or prevent the ocular surface disease. A “subject” as referred to herein, can be any mammal, such as a human. The subject has a corneal dysfunction or an ocular surface disease or is at risk of developing corneal dysfunction or an ocular surface disease.

Corneal dysfunction refers to a group of eye disorders including corneal endothelial dysfunction, acute or chronic corneal endothelial cell (CEC) loss, bullous keratopathy (PBK), Fuchs' endothelial dystrophy, corneal transplant failure, corneal transplant rejection, corneal inflammation, corneal edema, corneal degeneration, corneal melting, or corneal ectasia.

Ocular surface disease refers to a group of eye disorders including dry eye syndrome, meibomian gland dysfunction, blepharitis, ocular rosacea allergic conjunctivitis (including seasonal allergic conjunctivitis (SAC), perennial allergic conjunctivitis (PAC), atopic keratoconjunctivitis (AKC), vernal keratoconjunctivitis (VKC) and giant papillary conjunctivitis (GPC)), chemical and thermal ocular burns, pterygium, pinguecula, conjunctivochalasis or limbal stem cell deficiency.

### Inflammasome

Among the inflammatory responses, pyroptosis, a form of proinflammatory programmed cell death induced by inflammatory caspases, is activated and involved in keratocyte damage and pterygium formation¹²⁻¹⁴.

The inflammasome is a multiprotein complex of the innate immune response involved in the activation of inflammatory caspases such as caspase-1 and caspase-5 (caspase-11 in rodents), the processing of the pro-inflammatory cytokines IL-1β and IL-18, and the cell death process of pyroptosis¹⁵. The inflammasome is comprised of three main elements: the adaptor protein apoptosis-associated speck-like protein containing a caspase recruitment domain (ASC); a nucleotide oligomerization domain-like receptor (NLRs), which serves as a sensor that facilitates the proteolytic cleavage of caspases; and procaspase-1 which is activated upon inflammasome assembly^{16,17}. Pyroptosis involves the activation of GSDM-D that upon cleavage by caspases, inserts its amino terminus (GSDM-D-N) into the cell membrane to form 1 to 2 nm transmembrane pores, leading to cytoplasmic swelling and osmotic lysis with the subsequent release of inflammatory mediators and intracellular contents, including IL-1β¹⁸⁻²² See Arend et al. 2008; Li et al. 2008; and Martinon et al. 2002, each of which are incorporated by reference in their entireties.

### Inflammasome Inhibitors

In various embodiments, the inflammasome inhibitor is an agent which inhibits expression of one or more components of the inflammasome (e.g., an agent that inhibits the expression of ASC). By “inhibition” of expression is meant a reduction of expression compared to basal/wild-type levels (in the absence of treatment as described herein). It will be appreciated that inhibiting expression of one or more inflammasome related genes does not require 100% abolition of expression and/or protein production; any reduction in expression of one or more components of the inflammasome is contemplated and beneficial to a subject. For example, in various aspects, an inhibitor may reduce the expression of a component of an inflammasome (e.g., ASC) by 1, 5, 10, 15, 20, 25, 30, 40, 50, 60, 70, 80 90 or 100%. Alternatively, the inflammasome inhibitor does not interfere with expression, but inhibits the function or activity of one or more components of the inflammasome complex. Activity of the inflammasome complex may be determined using any suitable assay, such as the assays described in the Examples. Complete abrogation of activity is not required; an inhibitor may reduce one or more activities of the inflammasome (or a component thereof) by 1, 5, 10, 15, 20, 25, 30, 40, 50, 60, 70, 80 90 or 100%, so long as a beneficial effect is achieved in the cornea or ocular surface tissues.

In various embodiments, the inflammasome inhibitor is a polynucleotide.

Polynucleotides are typically delivered to a host cell via an expression vector, which includes the regulatory sequences necessary for delivery and expression. In some aspects, the constructs described herein include a promoter (e.g., cytomegalovirus (CMV) promoter), a protein coding region (optionally with noncoding (e.g. 3′-UTR) regions that facilitate expression), transcription termination sequences, and/or regulator element sequences. In various aspects, tissue specific or regulatable expression may be desired. In this regard, for example, the Cre-loxP system may be utilized to express a polynucleotide of interest (e.g., siRNA targeting the ASC gene).

Expression vectors may be viral-based (e.g., retrovirus-, adenovirus-, or adeno-associated virus based) or non-viral vectors (e.g., plasmids). Non-vector based methods (e.g., using naked DNA, DNA complexes, etc.) also may be employed. Optionally, the vector is a viral vector, such as a lentiviral vector or baculoviral vector, and in various preferred embodiments the vector is an adeno-associated viral vector (AAV). The expression vector may be based on any AAV serotype, including AAV-1, AAV-2, AAV-3, AAV-4, AAV-5, AAV-6, AAV-7, AAV-8, AAV-9, AAV-10, AAV-11, AAV-12, or AAV-13. Polynucleotides also may be delivered via liposomes, nanoparticles, exosomes, microvesicles, hydrodynamic-based gene delivery, and the like.

Exemplary inhibitory polynucleotides include, but are not limited to, antisense oligonucleotides (ASO), short hairpin RNA (shRNA), small interfering RNA (siRNA), or micro RNA (miRNA). In some embodiments, the inflammasome inhibitor is an siRNA, shRNA, ASO, or miRNA that inhibits the expression of caspase-1, ASC, NLRP1, NLRP2, NLRP3, or AIM2. For example, in various aspects, the agent is an antisense oligonucleotide (ASO) which knocks-down (i.e., reduces) the expression of the one or more components of the inflammasome. In exemplary aspects, the inflammasome component is ASC. An ASO is a single-stranded deoxyribonucleotide, which is complementary to an mRNA target sequence. In various aspects, the inflammasome antisense oligonucleotide targets an exonic or intronic sequence of one or more inflammasome gene(s). Suitable ASO sequences targeting, e.g., ASC are known in the art. For example, antisense oligonucleotides of ASC are described in Masumoto et al., J Biol Chem. 1999; 274(48):33835-8 (incorporated by reference).

RNA interference (RNAi) is a mechanism of gene regulation in eukaryotic cells that has been considered for the treatment of various diseases. As an understanding of natural RNAi pathways has developed, researchers have designed artificial siRNAs and shRNAs and for use in regulating expression of target genes for treating disease. Several classes of small RNAs are known to trigger RNAi processes in mammalian cells, including short (or small) interfering RNA (siRNA), short (or small) hairpin RNA (shRNA), and microRNA (miRNA). See, for example, Davidson et al., Nat. Rev. Genet. 12:329-40, 2011; Harper, Arch. Neurol. 66:933-8, 2009.

In various aspects, the inflammasome inhibitor is a small interfering RNA (siRNA), which knocks-down (i.e., reduces) the expression of the one or more components of the inflammasome (e.g., ASC, NLRP1, NLRP2, NLRP3, and/or AIM2). The siRNA can include an RNA strand (the antisense strand) having a region which is generally 30 nucleotides or less in length, e.g., 15-30 nucleotides in length or 19-24 nucleotides in length, which region is substantially complementary to at least part the targeted mRNA transcript. In certain embodiments, contacting a cell with the siRNA inflammasome inhibitor results in a decrease in the target mRNA level in a cell by at least about 5%, about 10%, about 20%, about 30%, about 40%, about 50%, about 60%, about 70%, about 80%, about 90%, about 95%, about 99%, up to and including 100% (e.g., a decrease in the amount of RNA encoding ASC or another inflammasome component) compared to levels found in a cell not exposed to the siRNA. The siRNAs may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

In some embodiments, the siRNA inflammasone inhibitor comprises a nucleotide sequence set forth in the Table below.

| | Sense strand | Antisense |
|---|---|---|
| | (5′->3′) | (5′->3′) |
| siRNA1 | ACAAGCUGGU | UAGAAGCUGA |
| | CAGCUUCUA | CCAGCUUGU |
| | (SEQ ID NO: 1) | (SEQ ID NO: 2) |
| siRNA2 | GACAAGCUGG | AGAAGCUGAC |
| | UCAGCAAGA | CAGCUUGUCN |
| | SEQ ID NO: 3) | N |
| | | (SEQ ID NO: 4) |
| siRNA3 | GACAAGCUGG | AGAAGCUGAC |
| | UCAGCUUCUN | CAGCUUGUCN |
| | (SEQ ID NO: 5) | (SEQ ID NO: 6) |
| siRNA4 | GACAAGCUGG | AGAAGCUGAC |
| | UCAGCUUCU | CAGCUUGUC |
| | (SEQ ID NO: 7) | (SEQ ID NO: 8) |
| siRNA5 | ACAAGCUGGU | UAGAAGCUGA |
| | CAGCUAGAU | CCAGCUUGUN |
| | (SEQ ID NO: 9) | N |
| | | (SEQ ID NO: 10) |
| siRNA6 | GACAAGCUGG | AGAAGCUGAC |
| | UCAGCUUCA | CAGCUUGUCN |
| | (SEQ ID NO: 11) | N |
| | | (SEQ ID NO: 12) |
| siRNA7 | CUCACCGACA | UGACCAGCUU |
| | AGCUGGUCA | GUCGGUGAGG |
| | (SEQ ID NO: 13) | U |
| | | (SEQ ID NO: 14) |
| siRNA8 | GACAAGCUGG | AGAAGCUGAC |
| | UCAGCUUCU | CAGCUUGUCG |
| | (SEQ ID NO: 15) | G |
| | | (SEQ ID NO: 16) |
| siRNA9 | UCACCGACAA | CUGACCAGCU |
| | GCUGGUCAG | UGUCGGUGAG |
| | (SEQ ID NO: 17) | G |
| | | (SEQ ID NO: 18) |
| siRNA10 | CACCGACAAG | GCUGACCAGC |
| | CUGGUCAGC | UUGUCGGUGA |
| | (SEQ ID NO: 19) | G |
| | | (SEQ ID NO: 20) |
| siRNA11 | ACCGACAAGC | AGCUGACCAG |
| | UGGUCAGCU | CUUGUCGGUG |
| | (SEQ ID NO: 21) | A |
| | | (SEQ ID NO: 22) |
| siRNA12 | CCGACAAGCU | AAGCUGACCA |
| | GGUCAGCUU | GCUUGUCGGU |
| | (SEQ ID NO: 23) | G |
| | | (SEQ ID NO: 24) |
| siRNA13 | CGACAAGCUG | GAAGCUGACC |
| | GUCAGCUUC | AGCUUGUCGG |
| | (SEQ ID NO: 25) | U |
| | | (SEQ ID NO: 26) |

Another exemplary inflammasome inhibitor used to inhibit the expression of one or more components of the inflammasome (e.g., ASC) may comprise components employed in genome-editing techniques, such as designer zinc fingers nucleases (ZFNs), transcription activator-like effectors nucleases (TALENs), or CRISPR-Cas (clustered regularly interspaced short palindromic repeats-CRISPR associated) systems. In some embodiments, the agents are used to modify the sequence of one or more genes of the inflammasome. In various aspects, the coding region or a regulatory element and/or non-coding region associated with the inflammasome genes is modified using the genome-editing. In various aspects, genome editing may be used to replace part or all of an inflammasome-related gene sequence or alter associated protein expression levels. An exemplary agent for use in the method of the disclosure is DNA encoding Cas9 molecules and/or guide RNA (gRNA) molecules. Cas9 and gRNA can be present in a single expression vector or separate expression vectors. Adenoviral delivery of the CRISPR/Cas9 system is described in Holkers et al., Nature Methods (2014), 11(10):1051-1057 which is incorporated by reference in its entirety. In related embodiments, the inflammasome inhibitor is an CRISPR Cas9 protein and one or more guide RNA molecules, TALEN or zinc finger nuclease (ZFN) targeting caspase-1, ASC, NLRP1, NLRP2, NLRP3, or AIM2.

In various embodiments, the inflammasome inhibitor is an antibody or an antigen-binding fragment thereof. Antibodies include, but are not limited to, polyclonal antibodies, monoclonal antibodies (mAbs), chimeric antibodies, or humanized antibodies The antibody may be of any immunoglobulin class including IgG, IgM, IgE, IgA, GILD or any subclass thereof. Antibody fragments include F(ab′)2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see, e.g., Hollinger and Hudson, Nature Biotechnology, 23(9):1126-1136 (2005)). An antibody may bind to its target with an affinity of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M. Affinity may be determined by an affinity ELISA assay. In certain embodiments, affinity may be determined by a BIAcore assay. In certain embodiments, affinity may be determined by a kinetic method. In certain embodiments, affinity may be determined by an equilibrium/solution method. Methods for determining monoclonal antibody specificity and affinity by competitive inhibition can be found in Harlow, et al., Antibodies: A Laboratory Manual, Cold Spring Harbor Laboratory Press, Cold Spring Harbor, N.Y., 1988, Colligan et al., eds., Current Protocols in Immunology, Greene Publishing Assoc. and Wiley Interscience, N.Y., (1992, 1993), and Muller, Meth. Enzymol. 92:589-601, 1983, which references are entirely incorporated herein by reference.

Monoclonal antibodies that specifically bind inflammasome components may be obtained by methods known to those skilled in the art. See, for example Kohler and Milstein, Nature 256:495-497, 1975: U.S. Pat. No. 4,376,110; Ausubel et al., eds., Current Protocols in Molecular Biology, Greene Publishing Assoc. and Wiley Interscience, N.Y., (1987, 1992); Harlow and Lane ANTIBODIES: A Laboratory Manual Cold Spring Harbor Laboratory Press, Cold Spring Harbor, N.Y., 1988; Colligan et al., eds., Current Protocols in Immunology, Greene Publishing Assoc. and Wiley Interscience, N.Y., (1992, 1993), the contents of which are incorporated entirely herein by reference.

Antibodies which inhibit the inflammasome (e.g., ASC antibodies and AIM2 antibodies) have been described in U.S. Pat. Nos. 8,685,400 and 10,703,811 (incorporated by reference in their entireties).

In various embodiments, the inflammasome inhibitor is an antibody or an antigen-binding fragment thereof that binds a mammalian ASC protein such as, for example, a human, mouse or rat ASC protein. The human ASC protein sequence is available as accession number NP_037390.2 (Q9ULZ3-1), NP_660183 (Q9ULZ3-2) or Q9ULZ3-3. The rat ASC protein is available as accession number NP_758825 (BAC43754). The mouse ASC protein is available as accession number NP_075747.3.

Any suitable anti-ASC antibody can be used, and several are commercially available. Examples of anti-ASC antibodies are described in U.S. Pat. No. 8,685,400, the contents of which are herein incorporated by reference in its entirety. Examples of commercially available anti-ASC antibodies include, but are not limited to IC 100 Anti-ASC from Zyversa therapeutics, 04-147 Anti-ASC, clone 2E1-7 mouse monoclonal antibody from Millipore Sigma, AB3607—Anti-ASC Antibody from Millipore Sigma, orb194021 Anti-ASC from Biorbyt, LS-C331318-50 Anti-ASC from LifeSpan Biosciences, AF3805 Anti-ASC from R & D Systems, NBP1-78977 Anti-ASC from Novus Biologicals, 600-401-Y67 Anti-ASC from Rockland Immunochemicals, D086-3 Anti-ASC from MBL International, AL177 anti-ASC from Adipogen, monoclonal anti-ASC (clone o93E9) antibody, anti-ASC antibody (F-9) from Santa Cruz Biotechnology, anti-ASC antibody (B-3) from Santa Cruz Biotechnology, ASC polyclonal antibody—ADI-905-173 from Enzo Life Sciences, or A161 Anti-Human ASC-Leinco Technologies.

In various embodiments, the inflammasome inhibitor is an antibody or an antigen-binding fragment thereof that binds to a mammalian AIM2 protein such as, for example, a human, mouse or rat AIM2 protein. The human AIM2 protein sequence is available as accession number NX_014862, NP004824, XP016858337, XP005245673, AAB81613, BAF84731 or AAH10940. Any suitable anti-AIM2 antibody can be used, and several are commercially available. Examples of commercially available anti-AIM2 antibodies include, but are not limited to a rabbit polyclonal anti-AIM2 cat. Number 20590-1-AP from Proteintech, Abcam anti-AIMS antibody (ab119791), rabbit polyclonal anti-AIM2 (N-terminal region) Cat. Number AP3851 from ECM biosciences, rabbit polyclonal anti-ASC Cat. Number E-AB-30449 from Elabsciences, Anti-AIM2 mouse monoclonal antibody called AIM2 Antibody (3C4G11) with catalog number sc-293174 from Santa Cruz Biotechnology, mouse monoclonal AIM2 antibody with catalog number TA324972 from Origene, AIM2 monoclonal antibody (10M2B3) from Thermofisher Scientific, AIM2 rabbit polyclonal antibody ABIN928372 or ABIN760766 from Antibodies-online, Biomatix coat anti-AIM2 polyclonal antibody with cat. Number CAE02153. Anti-AIM2 polyclonal antibody (0ABF01632) from Aviva Systems Biology, rabbit polyclonal anti-AIM2 antibody LS-C354127 from LSBio-C354127, rabbit monoclonal anti-AIM2 antibody from Cell Signaling Technology, with cat number MA5-16259. Rabbit polyclonal anti-AIM2 monoclonal antibody from Fab Gennix International Incorporated, Cat. Number AIM2 201AP, MyBiosource rabbit polyclonal anti-AIM2 cat number MBS855320, Signalway rabbit polyclonal anti AIM2 catalog number 36253, Novus Biological rabbit polyclonal anti-AIM2 catalog number 43900002, GeneTex rabbit polyclonal anti-AIM2 GTX54910, Prosci, rabbit polyclonal anti-AIM2 26-540, Biorbyt mouse monoclonal anti-AIM2 orb333902, Abcam rabbit polyclonal anti-AIM2 ab93015), Abcam rabbit polyclonal anti-AIM2 ab76423, Sigma Aldrich mouse polyclonal anti-AIM2 SAB1406827, or Biolegend anti-AIM2 3B10.

In various embodiments, the method comprises administering a combination of the foregoing inflammasome inhibitors, in a single composition or in separate compositions (optionally administered at different time points).

### Treatment or Prevention of Corneal Dysfunction or Ocular Surface Disease

The terms “treating” or “treatment” refer to reducing, delaying or ameliorating corneal dysfunction, ocular surface disease, associated disorders, and/or symptoms associated therewith. In various aspects, “treating” includes slowing, delaying, or halting the progression or incidence of cell death or dysfunction in a target tissue, thereby resulting in an improvement or stabilization of a disease or disorder. The terms “prevent” or “preventing” refers to slowing or delaying the onset of a disease or dysfunction or symptom associated therewith (e.g., corneal dysfunction, ocular surface disease, and/or associated disorders and/or symptoms associated therewith), or reducing or minimizing the symptoms of a disease or dysfunction upon onset. It is appreciated that, although not precluded, “treating” or “treatment” of a disorder or condition does not require that the disorder, condition, or symptoms be completely eliminated. Similarly, “prevent” or “preventing” a disorder or condition does not require 100% protection from that disorder, condition, or symptom. Any degree of improvement in a condition, stabilization of a condition, or inhibition/slowing of the onset of a condition, is contemplated. By the phrase “effective amount” is meant an amount sufficient to produce a therapeutically (e.g., clinically) desirable result; the exact nature of the result will vary depending on the nature of the corneal dysfunction or ocular surface disease being treated.

Exemplary methods of detecting or quantifying corneal dysfunction or ocular surface disease include, but are not limited to, corneal pachymetry, corneal topography, biomicroscopy, specular microscopy, confocal microscopy and optical coherence tomography. These methods are useful in characterizing the function of, e.g., the corneal endothelium, and are appropriate for characterizing treatment or prevention of an ocular disease, disorder, or condition.

In some embodiments, the methods comprise determining an elevated level of caspase-1 in a tear sample from the subject before the administering step. In some embodiments, an elevated level of caspase-1 in the tear sample compared to a control identifies the subject as likely to be suffering from ocular surface damage. In some embodiments, the control sample is a tear sample from a healthy subject. In some embodiments, the ocular surface damage is corneal staining. In some embodiments, the elevated level of caspase-1 in the tear sample comprises an amount that is at least two standard errors higher than an amount of caspase-1 in the tear sample from the control subject.

In some embodiments, an amount of caspase-1 in the tear sample at a level greater than 80 pg/mL identifies the subject as likely to be suffering from ocular surface damage. In some embodiments, the tear sample comprises caspase-1 at a level greater than 82 pg/mL (e.g., 83 pg/mL, 84 pg/mL, 85 pg/mL, 86 pg/mL, 87 pg/mL, 88 pg/mL, 89 pg/mL, 90 pg/mL, 100 pg/mL, 110 pg/mL, 120 pg/mL, 130 pg/mL, 140 pg/mL, 150 pg/mL, 160, pg/mL, 170 pg/mL, 180 pg/mL, 190 pg/mL, or 200 pg/mL). In some embodiments, an amount of caspase-1 in the tear sample that is at least two standard errors higher than the amount of caspase-1 in the tear sample from the control subject.

In some embodiments, described herein is a method of diagnosing ocular surface damage in a mammalian subject in need thereof, the method comprising: determining a level of caspase-1 in a tear sample from the subject, wherein an elevated level of caspase-1 in the tear sample compared to a control identifies the subject as likely to be suffering from ocular surface damage. In some embodiments, an amount of caspase-1 in the tear sample that is at least two standard errors higher than the amount of caspase-1 in the tear sample from the control subject, identifies the subject as likely to be suffering from ocular surface damage. In some embodiments, caspase-1 in the tear sample at a level greater than 80 pg/mL identifies the subject as likely to be suffering from ocular surface damage. In some embodiments, caspase-1 in the tear sample at a level greater than 81 pg/mL identifies the subject as likely to be suffering from ocular surface damage. In some embodiments, the tear sample comprises caspase-1 at a level greater than 82 pg/mL (e.g., 83 pg/mL, 84 pg/mL, 85 pg/mL, 86 pg/mL, 87 pg/mL, 88 pg/mL, 89 pg/mL, 90 pg/mL, 100 pg/mL, 110 pg/mL, 120 pg/mL, 130 pg/mL, 140 pg/mL, 150 pg/mL, 160, pg/mL, 170 pg/mL, 180 pg/mL, 190 pg/mL, or 200, pg/mL), identifying the subject as likely suffering from ocular surface damage. In some embodiments, the method further comprises administering an inflammasome inhibitor to the subject in an amount effective to treat the ocular surface damage. In some embodiments, the inflammasome inhibitor is an siRNA. In some embodiments, the inflammasome inhibitor is an antibody.

### Administration of Inflammasome Inhibitors

A dose of an active agent (inflammasome inhibitor) will depend on factors such as route of administration (e.g., local vs. systemic), patient characteristics (e.g., gender, weight, health, side effects), the nature and extent of the corneal dysfunction, ocular surface disease, or associated disorder, and the particular inflammasome inhibitor or combination of inflammasome inhibitors selected for administration.

Suitable methods of administering a physiologically-acceptable composition, such as a pharmaceutical composition comprising an agent described herein, are well known in the art. In various aspects, more than one route can be used to administer one or more of the inflammasome inhibitors disclosed herein. A particular route can provide a more immediate and more effective reaction than another route. For the methods of treating and/or preventing corneal dysfunction or ocular surface disease, ocular administration of the inflammasome inhibitor is contemplated (although not required). Ocular administration of the inflammasome inhibitor may comprise periocular or intraocular injections (peribulbar, subconjunctival, intracorneal, intracameral, intravitreal, etc.) or topical application. If desired, the inhibitor may be administered using an ocular implant, nanoparticles, microparticles, eye drops, gelsaqueous opthamalic sprays, and/or electrodynamic ocular spray treatment. In one particular embodiment, the inflammasome inhibitor may administered by periocular/intraocular injection or topically such as in the form of an eye drop.

In various aspects, the inflammasome inhibitor is a polynucleotide, such as siRNA, which is administered as part of composition further comprising a diluent, excipient, or buffer. Pharmaceutically-acceptable diluents, excipients, or buffers are known in the art. See for example, Bruno et al. Adv Drug Deliv Rev. 2011; 63(13):1210-1226 (incorporated by reference in its entirety). In various embodiments, the polynucleotide (e.g., siRNA) is formulated in a buffer, e.g., acetate buffer, citrate buffer, prolamin buffer, carbonate buffer, phosphate buffer, or any combination thereof. Simply resuspending a siRNA in phosphate buffered saline (PBS) has been demonstrated to be sufficient to provide a vehicle useful for expression of the siRNA in vivo. In some embodiments, the siRNA is provided siRNA is provided in phosphate buffered saline (PBS). In some embodiments, the siRNA is provided in non-buffered solution. In one embodiment, siRNA is provided in water.

In various aspects, the inhibitor is provided via ocular administration in an ophthalmically acceptable formulation, i.e., a composition comprising excipients, emulsifiers, wetting agents, carriers or fillers that are suitable for application to the tissues of the eye and eye area. Ophthalmically acceptable compositions may comprise, for example, the polyethylene glycols designated 200, 300, 400 and 600, or Carbowax designated 1000, 1500, 4000, 6000 and 10000, complexing agents, such as disodium-EDTA or EDTA, antioxidants, such as ascorbic acid, acetylcysteine, cysteine, sodium hydrogen sulfite, butyl-hydroxyanisole, butyl-hydroxy-toluene; stabilizers, such as thiourea, thiosorbitol, sodium dioctyl sulfosuccinate or monothioglycerol; or other excipients, such as, for example, lauric acid sorbitol ester, triethanol amine oleate or palmitic acid ester. The inhibitor may be provided in a tear substitute, i.e., a composition which lubricates the ocular surface, often mimicking the consistency of endogenous tears.

Alternative methods for delivering an inflammasome inhibitor to a subject includes, e.g., through injection or infusion by intravenous, intraotic, intraperitoneal, intracerebral (intra-parenchymal), intracerebroventricular, intramuscular, intraocular, intraarterial, intraportal, intralesional, intramedullary, intrathecal, intraventricular, transdermal, subcutaneous, intraperitoneal, intranasal, enteral, topical, sublingual, urethral, vaginal, or rectal means; by controlled, delayed, sustained or otherwise modified release systems; by implantation devices; using nanoparticles; or as a conjugate.

The inhibitor is preferably administered as soon as possible after it has been determined that the subject is at risk for corneal dysfunction or ocular surface disease (e.g. before, during or after an intraocular or periocular surgery, due to history of intraocular or periocular surgery (e.g., glaucoma drainage implant), due to family history, injury) or has demonstrated corneal dysfunction or ocular surface disease (e.g., upon detection of clinical manifestation of corneal dysfunction or ocular surface disease).

It is contemplated that two or more inflammasome inhibitors may be administered as part of a therapeutic regimen. Alternatively or in addition, one or more of the inflammasome inhibitors may be administered with other therapeutics as part of a therapeutic regimen. The inflammasome inhibitors may be administered as a monotherapy or as a combination therapy with other treatments administered simultaneously or metronomically. The term “simultaneous” or “simultaneously” refers to administration of two inflammasome inhibitors within six hours or less (e.g., within three hours or within one hour each other). In this regard, multiple inflammasome inhibitors may be administered in the same composition or in separate compositions provided within a short period of time (e.g., within 30 minutes). The term “metronomically” means the administration of different inflammasome inhibitors at different times and at a frequency relative to repeat administration. Inflammasome inhibitors need not be administered at the same time or by the same route; preferably, in various embodiments, there is an overlap in the time period during which different active agents are exerting their therapeutic effect.

Additional aspects and details of the disclosure will be apparent from the following examples, which are intended to be illustrative rather than limiting.

### EXAMPLES

### General Methods

### Donor Corneal Tissue

Experiments were conducted in accordance with the tenets of the Declaration of Helsinki for biomedical research involving human tissue. Human corneo-scleral buttons obtained from cadaveric corneal tissue unsuitable for transplantation were procured through the Florida Lions Eye Bank (Miami, FL). Corneal tissue grafts preserved in Optisol GS (Bausch & Lomb; Rochester, New York, USA) with a storage time of 14 days or less were used. Preservation to assay time was less than 14 days. All experiments were performed in triplicate, each being repeated at least three times.

### Endothelial Graft Preparation

To simulate the effect of inflammation and oxidative stress on the corneal endothelium ex vivo, experiments using corneal endothelial grafts were used. Human corneal endothelial grafts were prepared by the Florida Lions Eye Bank (Miami, FL, USA). The pre-cut corneal thickness of the cornea was measured using optical coherence tomography (RTVue, Optovue, USA). The cornea was then mounted on an artificial anterior chamber (Moria, France). A microkeratome (Moria cBM ALK, France) with a 300 mm depth blade was passed over the tissue to obtain a posterior lamellar thickness of approximately 90-100 mm within the central 8 mm. An “s” orientation marking using gentian violet surgical skin marker was placed on the stromal surface of the posterior segment at the periphery. The cornea's anterior cap was then remounted on the posterior segment before the cornea was removed from the anterior chamber. The pre-cut tissue was then transferred to a standard punching block (Moria, France), with the endothelial side facing up. The tissue was trephined using a 9.5-mm punch. The cornea was then gently placed into a corneal viewing chamber with Optisol-GS (Bausch & Lomb; Rochester, New York) to allow examination. The cornea's posterior segment thickness was measured using optical coherence tomography (RTVue, Optovue, USA) to obtain post-cut thickness. The corneal endothelium was examined via specular microscopy (CellCheck, Konan Medical) to determine endothelial cell density. Human donor grafts with endothelial cell densities above 2000 cells/mm² were used for this study.

### Endothelial Graft Exposure to Proinflammatory Cytokines

Endothelial grafts were isolated from corneal tissue grafts stored in Optisol GS (Bausch & Lomb; Rochester, New York, USA) and transferred to 24-well plates in DMEM/F12 medium (Gibco, Invitrogen Thermo Fisher, Carlsbad, CA, USA) supplemented with 5% fetal bovine serum (Gibco, Invitrogen Thermo Fisher, Carlsbad, CA, USA) and incubated at 37° C. for 48 hours to stabilize the cells following hypothermic storage. Medium was replaced and endothelial grafts were treated with TNF-α (100 ng/ml, R&D Systems, Minneapolis, MN, USA) and IFN-γ (100 ng/ml, Systems, Minneapolis, MN, USA)³⁴ and were incubated at 37° C. for 48 hours. Caspase-1 inhibitor, Ac-YVAD-cmk (30 ng/ml, InvivoGen, San Diego, CA, USA) was used to inhibit the inflammasome in CEC.

### Enzyme-Linked Immunosorbent Assay (ELISA)

To study the expression of inflammasome signaling proteins in endothelial grafts, cells were treated with TNF-α and IFN-γ for 48 hours. Treated endothelial grafts were placed on 100 ml of lysis buffer with protease inhibitor cocktail (Sigma) and Caspase-1 and IL-13 levels were measure by SimpleStep ELISA™ kit (Abcam; Cambridge, UK) according to the manufacturer's instructions. Caspase-1 and IL-1β protein levels were determined by measuring the fluorescence intensity using a microplate reader (SpectraMax iD3, Molecular Device, San Jose, CA, USA) with excitation and emission wavelengths set at 485 and 535 nm, respectively. All experiments were performed in duplicate and in three independent assays.

### Immunofluorescence

Endothelial grafts were fixed with 4% paraformaldehyde for 10 minutes at room temperature and washed with PBS. For immunofluorescence staining, samples were permeabilized with 0.3% Triton X-100 in PBS for 15 minutes and blocked with Image-iT FX signal enhancer (Invitrogen ThermoFisher Scientific; Carlsbad, CA, USA) for 30 minutes at room temperature. Isotype-matched nonspecific IgG antibodies were used as controls. Anti-caspase-1 rabbit polyclonal antibody (1:100, Abcam, Cambridge, UK) and anti-GSDM-D rabbit polyclonal antibody (1:100, Novus Biologicals, Colorado, USA) were used as primary antibodies to identify pyroptosis and left overnight at 4° C., followed by incubation of secondary goat anti-rabbit AlexaFluor 488 antibody (1:1000 MolecularProbes, Thermo Fisher, Eugene, OR, USA) for 45 minutes at room temperature. 4′,6-diamino020phenylindole (DAPI) staining (1:10,000, Sigma, St. Louis MO, USA) was used to counterstain cell nuclei. Stained samples were examined using an inverted fluorescence microscope (BZ-X800 Analyzer, KEYENCE, Osaka, Japan). Images were analyzed using BZ-X-Analyzer software (KEYENCE, Osaka, Japan).

### Lactate Dehydrogenase (LDH) Release Assay

Cytotoxicity of CEC after TNF-α, IFN-γ treatment was assessed by LDH release assay. LDH release assay was measure using a CytoTox 96® Non-Radioactive Cytotoxicity Assay kit (Promega, Madison, WI, USA) performed according to the manufacturer's instructions as described in³⁵. The absorbance was measured using a microplate reader (SpectraMax iD3, Molecular Device, San Jose, CA, USA) with excitation and emission wavelengths set at 490 nm.

### Intracellular ROS Detection

Measurement of intracellular ROS levels was performed using a 2,7-dichlorofluorescein diacetate (DCF-DA) Cellular Reactive Oxygen Species Detection Assay Kit (Abcam, Cambridge, UK) according to the manufacturer's instructions as described in³⁶. Endothelial grafts were incubated with 10 μM DCF-DA at 37° C. for 45 min. Then, ROS level was determined by measuring the fluorescence intensity using a microplate reader (SpectraMax iD3, Molecular Device, San Jose, CA, USA) with excitation and emission wavelengths set at 485 and 535 nm, respectively. Tert-butyl hydrogen peroxide (TBHP) was used as the positive control. All experiments were performed in duplicate and in three independent assays.

### Statistical Analysis

Results were analyzed by GraphPad Prism® v. 8.04 (GraphPad Software, La Jolla, CA, USA) using a one-way analysis of variance (ANOVA) followed by Holm-Sidak's multiple comparisons test. Differences were considered statistically significant at the level of p-values ≤0.05. A comparison between two groups of variables was performed using two-sided t-tests.

### Example 1: TNF-α and IFN-γ Activate the Inflammasome in Corneal Endothelial Cells

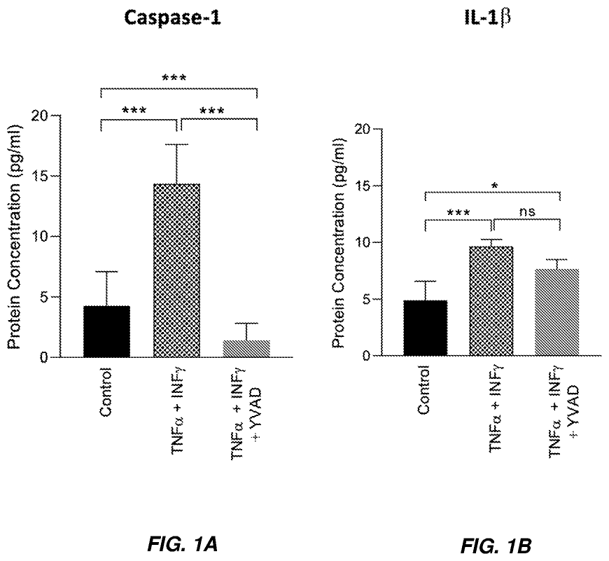

To determine the role of inflammation on CEC inflammasome activation, human corneal endothelial grafts exposed to proinflammatory cytokines, TNF-α and IFN-γ, and were analyzed for the protein levels of caspase-1 (FIG. 1A) and IL-1β (FIG. 1B). Caspase-1 levels were significantly elevated (p<0.0001) when compared to the control group, while cells treated with the caspase-1 inhibitor, Ac-YVAD-cmk, showed significantly lower levels of caspase-1 in comparison to exposed (TNF-α and IFN-γ) and control non-exposed endothelial grafts (p<0.0001) (FIG. 1A). Similarly, IL-1β levels were also significantly elevated (p<0.001), although grafts treated with the caspase-1 inhibitor Ac-YVAD-cmk did not show significantly lower levels of IL-1β in comparison to exposed (TNF-α and IFN-γ) endothelial grafts (FIG. 1B).

Next, caspase-1 expression was measured in human CEC exposed to proinflammatory cytokines TNF-α (100 ng/ml), IFN-γ (100 ng/ml) and caspase-1 inhibitor, Ac-YVAD-cmk (30 ng/ml) for 48 hours. Caspase-1 immunofluorescence was increased in corneal endothelial grafts grown in culture, while grafts treated with Ac-YVAD-cmk showed a lower expression of caspase-1. Thus, these findings indicate that the inflammasome is activated in endothelial cells following an inflammatory stimulus such as TNF-α and IFN-γ.

### Example 2: TNF-α and IFN-γ Increase GSDM-D Expression in Corneal Endothelial Cells

GSDM-D is a substrate of the inflammasome-mediated cell death process of pyroptosis³⁷. Thus, to determine the effects of TNF-α and IFN-γ-induced inflammation on CEC, corneal grafts were grown in culture and immunofluorescently stained with an antibody against GSDM-D following exposure to TNF-α (100 ng/ml) and IFN-γ (100 ng/ml) and/or caspase-1 inhibitor, Ac-YVAD-cmk for 48 hours. A higher intracellular (cytoplasmic) immunoreactivity of GSDM-D was detected in endothelial cells exposed to the inflammatory cytokines TNF-α and IFN-γ when compared to the control group and the Ac-YVAD-cmk-treated group, indicating that the substrate of pyroptosis is increased in cells following an inflammatory insult that is inhibited by inflammasome inhibition with Ac-YVAD-cmk.

### Example 3: Inflammation-Mediated Cell Death is Regulated by the Inflammasome

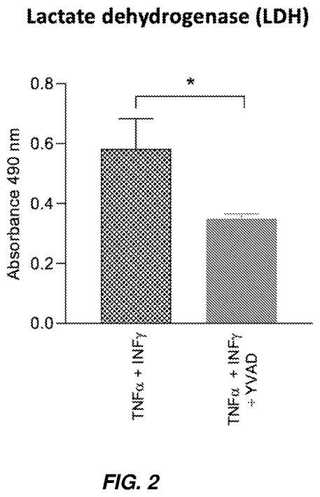

The inflammasome regulates the cell death process of pyroptosis. To assess the effects of inflammation in corneal cell death, LDH release on the corneal endothelial grafts exposed to TNF-α and IFN-γ, was measured. Furthermore, in order to assess whether the cell death process was mediated by the inflammasome, the caspase-1 inhibitor Ac-YVAD-cmk, was also used in corneal endothelial grafts exposed to TNF-α and IFN-γ (FIG. 2). LDH release was measured in the supernatant of corneal endothelial grafts exposed to TNF-α and IFN-γ. Accordingly, LDH levels were significantly downregulated (p<0.05) by Ac-YVAD-cmk indicating that cell death was decreased when the inflammasome was inhibited, thus indicating that the cell death process occurring in endothelial cells was in part pyroptosis.

### Example 4: Oxidative Stress Activates the Inflammasome in Corneal Endothelial Cells

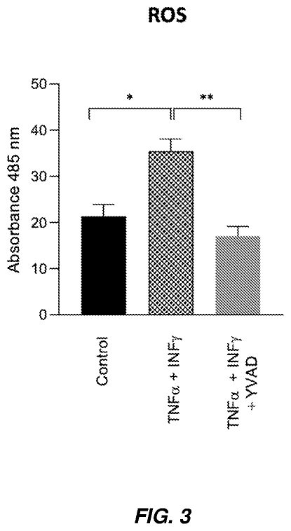

ROS are known to activate the inflammasome³⁸; thus, to test the effects of oxidative stress on inflammasome activation in corneal endothelial cells following an inflammatory insult by TNF-α and IFN-γ, ROS were measured in endothelial grafts in culture with and without caspase-1 inhibition with Ac-YVAD-cmk. Corneal endothelial cells exposed to TNF-α and IFN-γ showed significantly higher levels of ROS (p<0.05) (FIG. 3). Furthermore, ROS levels were significantly downregulated by caspase-1 inhibitor (p<0.001), showing that inflammasome inhibition has the potential to decrease the oxidative stress that is characteristic of the inflammatory response associated with corneal degeneration.

### Example 5: Clinical Data for ASC and IL-18 Levels in Patients with PBK

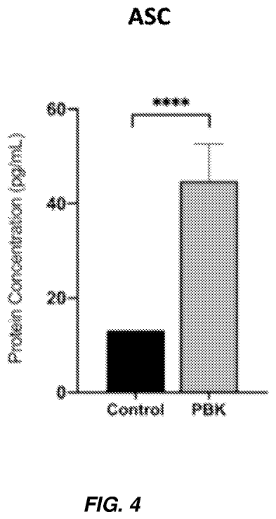

Next, ASC protein levels were measured in the aqueous humor of human patients with Pseudophakic bullous keratopathy (PBK) (FIG. 4). ASC protein levels in PBK patients undergoing corneal transplantation were compared to healthy patients undergoing standard phacoemulsification cataract surgery. ASC protein levels in the aqueous humor were found to be significantly elevated in patients with PBK.

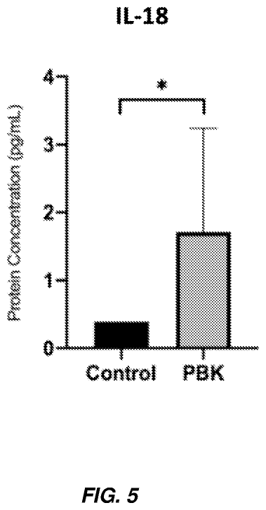

Levels of the pro-inflammatory inflammasome-derived cytokine, IL-18, were also measured in the aqueous humor of PBK patients undergoing corneal transplantation (FIG. 5). Similar to ASC protein levels, IL-18 protein levels in the aqueous humor were found to be significantly elevated in patients with PBK compared to healthy controls.

### Example 6: Clinical Data for ASC in Patients with Fuchs' Dystrophy and Failed Transplants

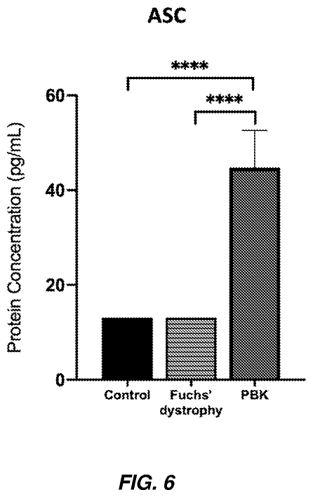

Next, ASC protein levels were measured in the aqueous humor of patients with Fuchs' dystrophy (FIG. 6). Surprisingly, ASC protein levels in the aqueous humor were not elevated in patients with Fuchs' dystrophy compared to ASC protein levels patients with PBK or healthy controls (aqueous humor was obtained from patients undergoing standard phacoemulsification cataract surgery right before surgery). This indicates that the chronic activation of the inflammasome was not secondary to corneal endothelial dysfunction, but to a history of intraocular surgery. Interestingly, although the mechanisms are still not well understood, it was confirmed that the inflammasome plays an important role in corneal endothelial dysfunction.

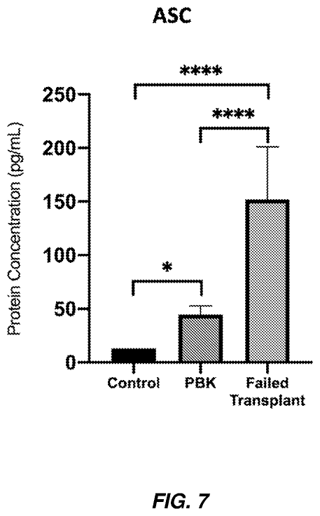

ASC protein levels were also elevated in patients with chronic corneal transplant failure compared to ASC protein levels in healthy controls (aqueous humor was obtained from patients undergoing standard phacoemulsification cataract surgery right before surgery) (FIG. 7). Failed transplants were patients undergoing a new corneal transplantation due to previous corneal transplant failure.

### Example 7: Role of Caspase-1 as a Biomarker of Ocular Surface Damage

The objective of the study was to evaluate relationships between caspase-1 and ocular surface metrics. It was hypothesized that caspase-1 levels would be most closely related to ocular surface damage, irrespective of etiology. Furthermore, the potential of tear caspase-1 levels as a biomarker for ocular surface damage to potentially aid in settings (e.g. primary health offices) where a slit lamp is not available was evaluated. Given the availability of the InflammaDry®, a point of care test which qualitatively assesses for ocular surface levels of Matrix Metalloproteinase-9 (MMP-9), its potential as a biomarker of ocular surface damage was examined and its results were compared to tear caspase-1. The use of anti-inflammatory therapies and their impact on tear caspase-1 level was also evaluated.

Study population: The first case group included individuals with dry eye disease (DED), which was defined as Ocular Surface Disease Index (OSDI)≥13 (Miller, K. L., et al., Arch Ophthalmol, 2010. 128(1): p. 94-101) and/or CS≥3. (Huang, J. F., et al., Invest Ophthalmol Vis Sci, 2012. 53(8): p. 4556-64; Pellegrini, M., et al., Transl Vis Sci Technol, 2019. 8(6): p. 34; Sook Chun, et al., Am J Ophthalmol, 2014. 157(5): p. 1097-102). This broad inclusion criteria was intentional as the aim was to include a wide range of DED symptoms and signs.

The second case group included any individuals who used glaucoma medications, irrespective of ocular surface symptoms and signs. Asymptomatic subjects (OSDI<13 points) with no signs ocular surface damage (CS<3 points on NEI scale) were enrolled into the control group. Exclusion criteria included pregnancy and age younger than 21 or older than 90 years old. Based on these groupings, a total of 64 patients (113 eyes) were recruited (DED n=33 patients, 62 eyes; glaucoma n=20 patients, 32 eyes; controls n=11 patients, 20 eyes).

Data and tear sample collection: After signing informed consent, all individuals filled out questionnaires regarding demographics, past medical and ocular history, and use of eyedrops (artificial tears (ATs), anti-inflammatory, hypotensive medications). Individuals first filled out the OSDI and then underwent a clinical examination with the following assessments, in the order performed: eye bulbar redness using the Effron bulbar redness scale (0=no hyperemia to 4=severe hyperemia) (Efron, N., Ophthalmic Physiol Opt, 1998. 18(2): p. 182-6); MMP-9 (InflammaDry®, graded as presence or absence of a pink strip) (Lanza, N. L., et al., Ocul Surf, 2016. 14(2): p. 189-95); TBUT, performed using fluorescein strips; CS with fluorescein (NEI scale, range 0-15 points) (Pellegrini et al., supra); conjunctival staining with lissamine green (NEI score ranges 0-18 points) (Begley, C., et al., Ocul Surf, 2019. 17(2): p. 208-220); and Schirmer's test with anesthesia. After 5 minutes, the Schirmer strips were removed with sterile gloves and placed in sterile 1.5 mL Eppendorf® containers.

Sample processing: Samples were processed as described by Dermer et al. with a modified technique. (Dermer, H., et al., J Clin Med, 2019. 8(10).) Briefly, after collection of the samples, the strips were immediately transferred from the 1.5 mL tubes to 0.5 mL sterile Eppendorf® tubes in which a small hole was previously cut towards the bottom. 60 microliters of balanced saline solution (BSS) were added to the 0.5 mL containers, which were placed inside 1.5 mL tubes. These were placed on a shaker at 400 rpm at room temperature (23° C.) for 30 minutes. Then, another 60 mL of BSS were further added and centrifuged at 10,000 rpm for 3 minutes. The supernatant was then collected from the 1.5 mL Eppendorf® tube and kept at −80° C. until their further processing with enzyme-linked immunosorbent assay (ELISA).

Caspase-1 Measurement: The concentration of caspase-1 in tears was determined using a commercially available ELISA kit (Abcam, Boston, MA), according to manufacturer's instructions. Briefly, tear supernatants and protein standards were loaded into different wells of a 96 well plate and mixed with an antibody cocktail followed by incubation for 1 hour at room temperature on a plate shaker set to 400 rpm. Then, the plate was washed with wash buffer solution. TMB substrate was added to each well and incubated in the dark on the plate shaker for minutes. Finally, the stop solution was added to each well and the absorbance was measured at 450 nm in a SpectraMax M5 (Molecular Devices) spectrophotometer.

Statistical analyses: R 3.6.1 (R Core Team, Vienna, Austria) was used for statistical analyses. Analyses were completed using linear mixed models (LMM) with random effects placed at the patient level to account for potential inter-eye correlations among those subjects with both eyes enrolled in the study. Data were adjusted for age and gender. Data are presented as mean±standard deviation (SD). P-values less than 0.05 were considered significant. GraphPad Prism 9.1.0 (216) (GraphPad Software Inc., La Jolla, CA) was used to generate figures.

Analysis of variance (ANOVA) and chi square were performed for comparison of demographic characteristics. Spearman correlations were used to determine if caspase-1 levels could reflect whether patients had ocular damage signs. The area under the Receiver Operating Characteristics (ROC) curve (AUC) of caspase-1 and InflammaDry® were then calculated to compare the biomarker potential of these two analytes in our study population. A LMM was used to determine whether using anti-inflammatory medications (cyclosporine, lifitegrast, or steroids) had an effect on tear caspase-1 levels.

Results:

Demographic characteristics and clinical parameters: This study included a total of 64 patients (113 eyes). A total 33 patients (62 eyes) belonged to the DED group, 20 patients (32 eyes) to the glaucoma group and 11 patients (20 eyes) to the control group. Demographic data is provided in Table 1.

TABLE 1
Demographic characteristics of the population and comorbidities

| | Controls | DED | Glaucoma | p-value |
|---|---|---|---|---|
| N | 11 | 33 | 20 | |
| Eyes (n) | 20 | 61 | 32 | |
| Age (mean ± SD) | 41 ± 13 | 57 ± 17 | 70 ± 10 | <0.0001 |
| Gender (Female, n (%)) | 7 (64%) | 28 (85%) | 9 (45%) | 0.009 |
| Race (n (%)) | | | | 0.65 |
| White | 9 (82%) | 30 (91%) | 18 (90%) | |
| Black | 2 (18%) | 2 (6%) | 1 (5%) | |
| Asian | 0 (0%) | 1 (3%) | 1 (5%) | |
| Ethnicity (n (%)) | | | | 0.35 |
| Non- Hispanic or Latino | 1 (9%) | 6 (18%) | 6 (30%) | |
| Hispanic or Latino | 10 (91%) | 27 (82%) | 14 (70%) | |
| Immunologic comorbidities | | | | |
| (n (%)) | | | | |
| Sjögrens Syndrome | 0 (0%) | 6 (18%) | 0 (0%) | |
| Rosacea | 0 (0%) | 4 (12%) | 0 (0%) | |
| Rheumatoid arthritis | 0 (0%) | 1 (13%) | 1 (5%) | |
| GVHD | 0 (0%) | 1 (3%) | 0 (0%) | |
| Bronchiectasis | 0 (0%) | 2 (6%) | 0 (0%) | |
| Fibromyalgia | 0 (0%) | 1 (3%) | 0 (0%) | |

DED: Dry Eye Disease; SD: Standard Deviation; GVHD: graft versus host disease.

There were statistically significant differences between age and sex distributions between the three groups. Data on topical medication use (per individual eye) are listed in Table 2.

TABLE 2
Ocular topical medications (per individual eye)

| Medications, n (%) | Controls | DED | Glaucoma |
|---|---|---|---|
| Cyclosporine | 0 (0%) | 11 (18%) | 1 (3%) |
| Prednisolone | 0 (0%) | 2 (3%) | 1 (3%) |
| Lifitegrast | 0 (0%) | 4 (7%) | 0 (0%) |
| Number of glaucoma medications | | | |
| 1 | 0 (0%) | 0 (0%) | 11 (34%) |
| 2 | 0 (0%) | 0 (0%) | 5 (16%) |
| 3 | 0 (0%) | 0 (0%) | 9 (28%) |
| 4 | 0 (0%) | 0 (0%) | 7 (22%) |

DED: Dry Eye Disease.

Mean values and SD of all assessed parameters are provided in Table 3.

TABLE 3

| Parameter | Controls | DED | Glaucoma |
|---|---|---|---|
| OSDI (mean ± SD) | 2.25 ± 2.46 | 47.92 ± 22.58 | 33.22 ± 28.13 |
| Bulbar redness (mean ± SD) | 0.80 ± 0.44 | 1.16 ± 0.38 | 2 ± 1.21 |
| MMP-9 (positive) | 35.29% | 37.70% | 53.33% |
| TBUT, seconds (mean ± SD) | 6.88 ± 2.95 | 5.44 ± 2.96 | 4.09 ± 2.65 |
| Corneal staining (mean ± SD) | 0.21 ± 0.41 | 2.13 ± 3.25 | 3.31 ± 2.19 |
| Conjunctival staining (mean ± SD) | 0.21 ± 0.42 | 1.20 ± 2.79 | 1.37 ± 1.51 |
| Schirmer's Test, mm wetting (mean ± SD) | 22.60 ± 10.95 | 15.23 ± 9.57 | 14.50 ± 8.08 |

DED: Dry eye disease;
OSDI Ocular Surface Disease Index;
MMP-9: Matrix metalloproteinase-9;
TBUT: tear break-up time;
SD: standard deviation

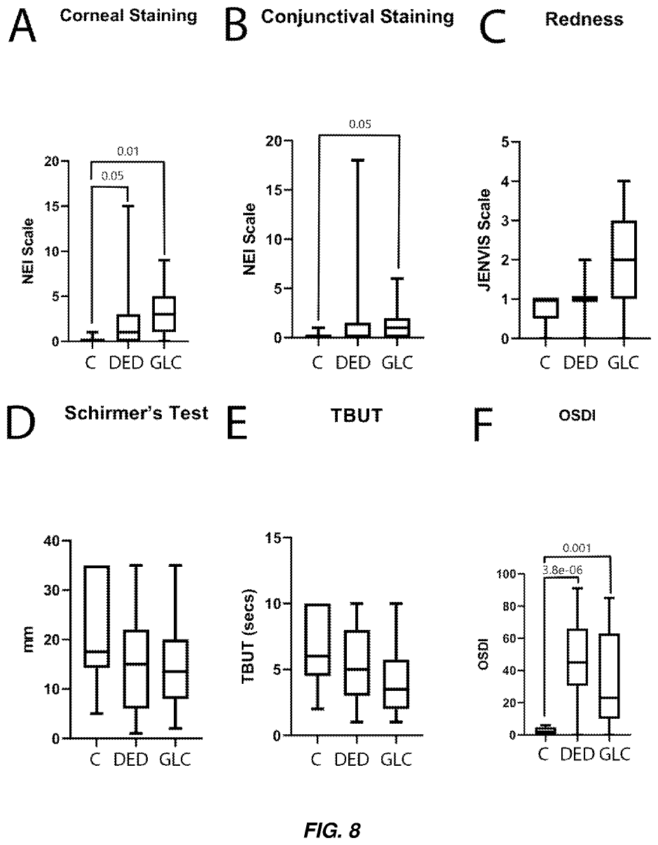

Overall, eyes receiving topical glaucoma medications had the most severe signs of ocular surface damage, with 66% of these individuals having CS≥3 (FIG. 8).

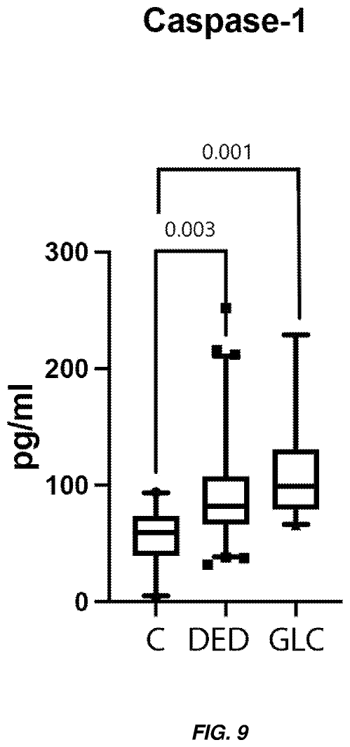

Comparison of tear caspase-1 levels between control, dry eye and glaucoma patients. Caspase-1 was significantly elevated in the glaucoma eyes, followed by the DED and control eyes (109.20±42.59 pg/mL, 91.62±43.86 pg/mL and 54.88±23.04 pg/mL, respectively) with significant differences observed between the glaucoma and DED eyes compared to controls (p=0.001 and 0.003 respectively; FIG. 9). Given differences in age and sex between groups, univariable and multivariable linear mixed models were conducted to examine caspase-1 levels among the study groups, while controlling for age, sex, and inter-eye correlations. Age and gender are not significantly associated with caspase-1 level. Even when adjusting for these potential confounders, the caspase-1 level remains significantly associated with the study group (Table 4).

TABLE 4
Coefficients from univariable and multivariable
linear mixed models predicting caspase-1 levels

| | Univariable | | Multivariable | |
|---|---|---|---|---|
| | Coefficient | p-value | Coefficient | p-value |
| Age (years) | 0.38 | 0.21 | −0.27 | 0.43 |
| Gender (male) | 9.85 | 0.39 | 8.32 | 0.46 |
| Study group | | | | |
| Control | 55.89 | 0.001 | — | — |
| Dry eye disease | 94.35 | 0.005 | 108.23 | 0.004 |
| Glaucoma | 109.28 | 0.0005 | 123.51 | 0.001 |

Whether caspase-1 level had a relationship with the number of topical medications used in the glaucoma group was also evaluated. While there was a positive trend, the association was not statistically significant (p=0.25; data not shown).

Caspase-1 levels are correlated with lack of tear production, tear instability and with ocular surface damage signs, but not with symptoms. A significant correlation between caspase-1 level and the following variables: CS (Spearman r=0.31, p=0.001), TBUT (Spearman r=−0.33, p=0.0006), Schirmer's test (Spearman r=−0.46, p=<0.0001) and bulbar redness (Spearman r=0.39, p=0.004), was identified. Neither conjunctival staining (Spearman r=0.19, p=0.06) nor symptoms assessed through OSDI (Spearman r=0.08, p=0.38) demonstrated correlation with caspase-1 level. (Table 5).

TABLE 5
Correlations between ocular surface damage signs
and symptoms and caspase-1 levels in tears.

| Variable | Spearman r | p-value |
|---|---|---|
| OSDI | 0.08 | 0.38 |
| Bulbar redness | 0.39 | 0.004 |
| TBUT | 0.33 | 0.0006 |
| Corneal Staining | 0.31 | 0.001 |
| Conjunctival staining | 0.19 | 0.06 |
| Schirmer's Test | 0.46 | <0.0001 |

OSDI: Ocular Surface Disease Index; TBUT: Tear Break-Up Time.

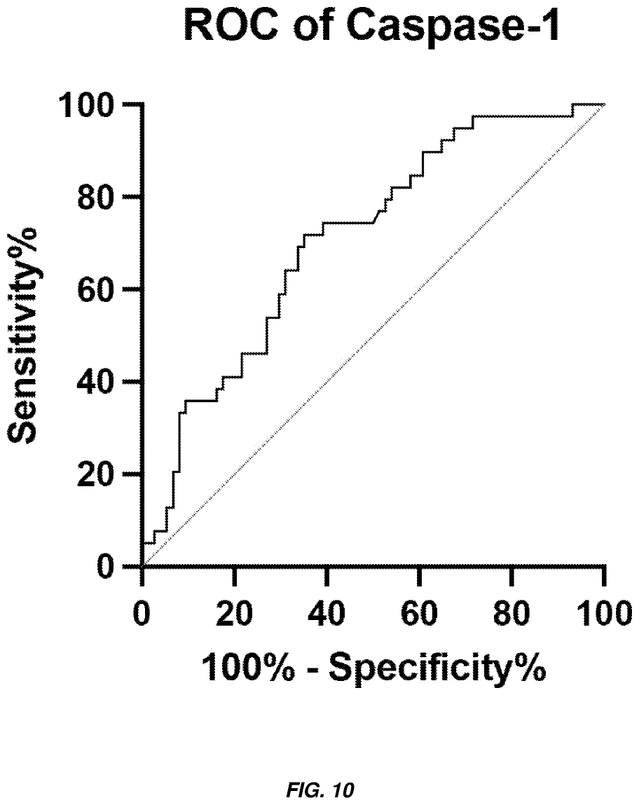

Caspase-1 as a biomarker of ocular surface damage. Next, the sensitivity and specificity of tear caspase-1 with CS≥3 points as the gold standard of ocular surface damage was examined. (FIG. 10). An AUC of 0.7 (p=0.0002, confidence interval: 0.62 to 0.81, SEM: 0.05) was found. A cutoff point of 82.85 pg/mL maximized the sensitivity (73%) and specificity (64%). A similar analysis was performed to examine InflammaDry® positivity as compared to CS≥3. Here, the AUC was 0.6 (p=0.04) with a positive InflammaDry® providing a sensitivity and specificity of 52% and 66%, respectively.

Relationships between caspase-1 in tears and topical anti-inflammatory medication use. Finally, relationships between the use of topical medications and caspase-1 levels in tears were examined. After conducting a multivariable linear mixed model in our population to adjust for age, sex, and potential inter-eye correlations, the tear caspase-1 level of individuals receiving anti-inflammatory eyedrops (cyclosporine, corticosteroids, or lifitegrast, n=16) were not different to those of individuals not using a topical anti-inflammatory agents (p=0.13).

Discussion:

It was determined that caspase-1, a molecule involved in the Inflammasome cascade, was elevated in individuals using topical anti-hypertensive medications and in those with a variety of ocular surface abnormalities, including corneal staining (CS), low tear production, and tear instability signs as compared to controls. Furthermore, caspase-1 was evaluated as a potential biomarker for corneal staining (CS), finding that tear caspase-1 levels ≥82.85 pg/ml had a 73% sensitivity of identifying individuals with clinically significant CS. In contrast, the sensitivity and specificity of InflammaDry® (which detects MMP-9 levels ≥40 ng/mL) had a lower sensitivity and specificity.

Overall, the data provided herein indicate that caspase-1 is a more reliable biomarker of corneal staining compared to InflammaDry®, that could be used as a screening test for ocular surface damage in clinics that do not have access to a slit lamp.

The data provided herein demonstrates that the inflammasome pathway is active in individuals with signs of ocular surface damage. Interestingly, caspase-1 levels correlated with signs of ocular surface damage, pointing to its potential as a diagnostic biomarker and therapeutic target. The measurement of caspase-1 may be useful as a point-of-care testing in primary care practices. It could be combined with the InflammaDry® for the care and follow up of patients with ocular surface damage or those receiving ocular medications, especially when they use anti-inflammatory agents that may suppress the expression of MMP-9.

### Example 8—Intraocular Inflammasome Activation in Patients with Corneal Graft Failure (CGF)

To determine intraocular inflammasome activation in patients with corneal graft failure (CGF), the AqH of these patients was collected to analyze the protein levels of commonly known pyroptosis markers. The aqueous humor (AqH) samples were collected under sterile conditions at the beginning of surgery from patients with corneal graft failure (CGF) (n=7), Fuchs' dystrophy (n=4) and pseudophakic bullous keratopathy (PBK) (n=5) who underwent corneal transplantation, as well as healthy patients who underwent cataract surgery (n=12). After corneal paracentesis, the AqH samples were obtained using a 27-gauge cannula. The samples were placed in 1.5 mL tubes and stored at −80° C. until analysis.

Inflammasome activation was assessed by measuring the levels of the inflammasome signaling proteins (The apoptosis-associated speck-like protein containing a caspase recruitment domain [ASC], interleukin [IL]-18, and IL-1β) in the AqH samples via Simple Plex technology (Protein Simple).

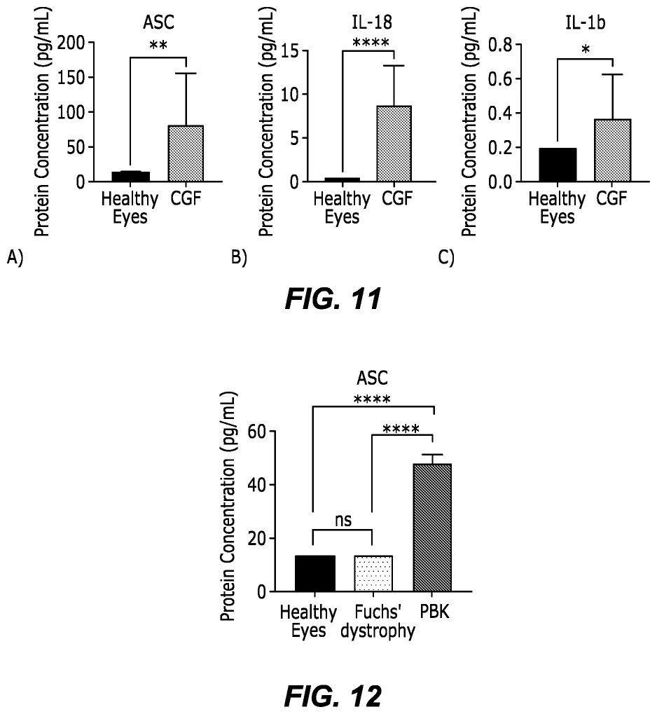

As shown in FIG. 11, the presence of inflammasome-mediated cell death markers differs significantly across the AqH of patients with CGF compared with healthy patients (Fisher's exact test p=<0.0001). It was determined that levels of ASC (FIG. 11A), IL-18 (FIG. 11B) and IL-1β (FIG. 11C) were significantly elevated in patients with CGF compared with the AqH of healthy patients. Additionally, it was determined that the levels of ASC were significant elevated in patients with PBK compared with Fuchs' dystrophy and healthy patients (FIG. 12).

### Example 9—Intracellular Expression of ASC in Corneal Graft Failure

Failed corneal grafts were collected from patients undergoing repeat corneal transplantation. The samples were placed in formalin. Samples were permeabilized with 0.3% Triton X-100 in PBS for 15 minutes and blocked with Image-iT FX signal enhancer (Invitrogen ThermoFisher Scientific; Carlsbad, CA, USA) for 30 min at room temperature. Isotype-matched nonspecific IgG antibodies were used as controls. Anti-caspase-1 rabbit polyclonal antibody (1:100, Abcam, Cambridge, UK) and anti-ASC mouse monoclonal antibody (1:100, Temecula, California, USA) were used as primary antibodies to identify pyroptosis and were left overnight at 4° C., followed by incubation of secondary goat anti-rabbit AlexaFluor 488 antibody and secondary goat anti-mouse AlexaFluor 555 antibody (1:200 MolecularProbes, Thermo Fisher, Eugene, OR, USA) for 45 min at room temperature. 4′,6-diamino020phenylindole (DAPI) staining (1:10,000, Sigma, St. Louis MO, USA) was used to counterstain cell nuclei. Stained samples were examined using an inverted fluorescence microscope (BZ-X800 Analyzer, KEYENCE, Osaka, Japan). Images were analyzed using BZ-X-Analyzer software (KEYENCE, Osaka, Japan).

Results showed an increased fluorescence intensity of ASC within the corneal samples (data not shown), which indicated an activation of pyroptosis in patients with corneal graft failure.

### Example 10—Inflammasome Activation in Corneal Endothelial Cells after Aqueous Humor Exposure

Human corneal endothelial cells (HCEC-B4G12) were cultured until they reached confluency. Then cells were exposed to 50 μl of aqueous humor (AqH) from patients with CGF or healthy patients, and cells with no exposure were used as a negative control. After 60 minutes of incubation in a humidified incubator under an atmosphere of 5% CO₂/95% air at 37° C., the aqueous humor was collected, and the cells were incubated with serum free medium (Human Endothelial SFM, Gibco) for 24 hours until protein extraction was performed for further analysis. This experiment was performed in triplicate.

Treated HCEC-B4G12 endothelial cells were placed on 100 μL of lysis buffer with protease inhibitor cocktail (Sigma), and caspase-1 was measured by SimpleStep ELISA™ kit (Abcam; Cambridge, UK) according to the manufacturer's instructions. Caspase-1 levels were determined by measuring the fluorescence intensity using a microplate reader (SpectraMax iD3, Molecular Device, San Jose, CA, USA) with excitation and emission wavelengths set at 485 and 535 nm, respectively. This experiment was performed in triplicate.

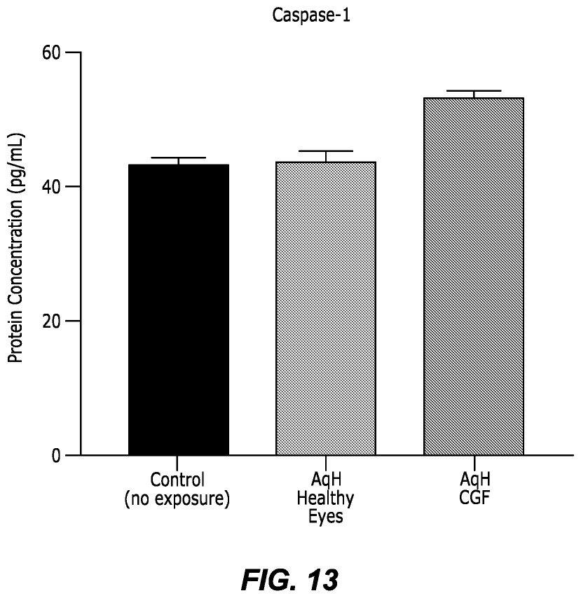

As shown in FIG. 13, caspase-1 was elevated in the cells treated with AqH from patients with CGF compared to the ones exposed to AqH from healthy eyes and the negative control group. Also, similar levels were seen in the cells exposed to the AqH of healthy eyes compared to the negative control group, suggesting that the inflammasome is not activated in the AqH of healthy eyes.

### Example 11—siRNA Inflammasome Inhibitor In Vitro Assay

HCEC-B4G12 (Human Corneal Endothelial Cell Line) cells were obtained from Leibniz Institute DSMZ and maintained in Endothelial SFM supplemented with FGF 10 ng/ml in a humidified incubator under an atmosphere of 5% CO2/95% air at 37° C. For transfection, cells were seeded in 12 well culture plates until cultures reached 95-100% confluence. The cells were then transfected with 1 μM ASC siRNAs using siPORT NeoFX Transfection Agent (Invitrogen) following the manufacturer protocol.

Transfection efficiency was estimated by assessing the amount of Block-it Alexa Fluor green fluorescent oligonucleotide (Invitrogen) present inside cells in control cultures 24 and 72 hours after transfection. After 48 hours of transfection to induce inflammation cells were treated with TNF-α (100 ng/ml, R&D Systems, Minneapolis, MN, USA) and IFN-γ (100ce ng/ml, Systems, Minneapolis, MN, USA). Endothelial cells were then incubated at 37° C. for 72 hrs. Following the 72 hours treated endothelial cells were placed on 150 μL of lysis buffer with protease inhibitor cocktail (Sigma), and caspase-1 levels were measured by SimpleStep ELISA™ kit (Abcam; Cambridge, UK) according to the manufacturer's instructions. Caspase-1 protein levels were determined by measuring the fluorescence intensity using a microplate reader (SpectraMax iD3, Molecular Device, San Jose, CA, USA) with excitation and emission wavelengths set at 485 and 535 nm, respectively. All experiments were performed 6 times. Results are provided in the table below.

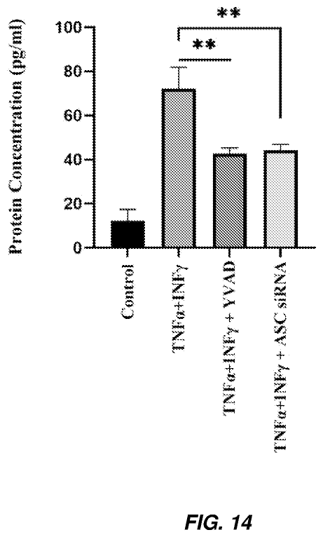

Caspase-1 levels were analyzed by SimpleStep ELISA after 72 hours of treatment with either YVAD (30 ng/ml) or ASC siRNA (1 μM) transfection, both sets of cells underwent inflammation induction with TNFα and INFγ. Results show means+/−SEM of six independent experiments. Statistical significance was calculated by unpaired Student's t-test**p<0.005. A significant decrease of about 38% of caspase-1 levels was observed on cells exposed to ASC siRNA compared to the positive control cells treated only with TNFα and INFγ, and a decrease of about 41% was observed in cells treated with YVAD. See FIG. 14. The preliminary results on this experiment showed that silencing ASC with siRNAs designed to specifically silence the adaptor receptor ASC, are capable of reducing caspase-1 levels, a molecule known to promote secretion of proinflammatory cytokines as well as pyroptosis in cells.

The present data shows that the efficacy of ASC siRNA in reducing caspase-1 levels is similar to that of YVAD, a selective irreversible inhibitor of caspase-1, with the added value that siRNAs are extremely precise molecules exclusively designed to silence expression of a target gene. Moreover, naked RNAs are quickly degraded and cleared by the kidneys, thus reducing the likelihood of systemic side effects.

### REFERENCES

1. Edelhauser H F. The balance between corneal transparency and edema: the Proctor Lecture. Invest Ophthalmol Vis Sci 2006; 47:1754-67.
2. Bourne W M. Clinical estimation of corneal endothelial pump function. Trans Am Ophthalmol Soc 1998; 96:229-39; discussion 39-42.
3. Polisetti N, Joyce N C. The culture of limbal stromal cells and corneal endothelial cells. Methods Mol Biol 2013; 1014:131-9.
4. Joyce N C. Proliferative capacity of corneal endothelial cells. Exp Eye Res 2012; 95:16-23.
5. Bourne R R, Minassian D C, Dart J K, Rosen P, Kaushal S, Wingate N. Effect of cataract surgery on the corneal endothelium: modern phacoemulsification compared with extracapsular cataract surgery. Ophthalmology 2004; 111:679-85.
6. Bourne W M, Nelson L R, Hodge D O. Continued endothelial cell loss ten years after lens implantation. Ophthalmology 1994; 101:1014-22; discussion 22-3.
7. Claerhout I, Beele H, Kestelyn P. Graft failure: I. Endothelial cell loss. Int Ophthalmol 2008; 28:165-73.
8. Bourne W M. Cellular changes in transplanted human corneas. Cornea 2001; 20:560-9.
9. Peh G S, Beuerman R W, Colman A, Tan D T, Mehta J S. Human corneal endothelial cell expansion for corneal endothelium transplantation: an overview. Transplantation 2011; 91:811-9.
10. Suzuki N, Yamaguchi T, Shibata S, et al. Cytokine Levels in the Aqueous Humor Are Associated With Corneal Thickness in Eyes With Bullous Keratopathy. Am J Ophthalmol 2019; 198:174-80.
11. Yamaguchi T, Higa K, Suzuki T, et al. Elevated Cytokine Levels in the Aqueous Humor of Eyes With Bullous Keratopathy and Low Endothelial Cell Density. Invest Ophthalmol Vis Sci 2016; 57:5954-62.
12. Niu L, Li L, Xing C, et al. Airborne particulate matter (PM2.5) triggers cornea inflammation and pyroptosis via NLRP3 activation. Ecotoxicol Environ Saf 2020; 207:111306.
13. Zheng T, Zhao C, Zhao B, et al. Impairment of the autophagy-lysosomal pathway and activation of pyroptosis in macular corneal dystrophy. Cell Death Discov 2020; 6:85.
14. Sun N, Zhang H. Pyroptosis in pterygium pathogenesis. Biosci Rep 2018; 38.
15. de Rivero Vaccari J P, Dietrich W D, Keane R W. Therapeutics targeting the inflammasome after central nervous system injury. Transl Res 2016; 167:35-45.
16. Martinon F, Burns K, Tschopp J. The inflammasome: a molecular platform triggering activation of inflammatory caspases and processing of prolL-beta. Mol Cell 2002; 10:417-26.
17. Srinivasula S M, Poyet J L, Razmara M, Datta P, Zhang Z, Alnemri E S. The PYRIN-CARD protein ASC is an activating adaptor for caspase-1. J Biol Chem 2002; 277:21119-22.
18. Shi J, Gao W, Shao F. Pyroptosis: Gasdermin-Mediated Programmed Necrotic Cell Death. Trends Biochem Sci 2017; 42:245-54.
19. Feng S, Fox D, Man S M. Mechanisms of Gasdermin Family Members in Inflammasome Signaling and Cell Death. J Mol Biol 2018; 430:3068-80.
20. Xia S, Ruan J, Wu H. Monitoring gasdermin pore formation in vitro. Methods Enzymol 2019; 625:95-107.
21. Kayagaki N, Stowe I B, Lee B L, et al. Caspase-11 cleaves gasdermin D for non-canonical inflammasome signalling. Nature 2015; 526:666-71.
22. Shi J, Zhao Y, Wang Y, et al Inflammatory caspases are innate immune receptors for intracellular LPS. Nature 2014; 514:187-92.
23. Dai C, Krantz S B. Interferon gamma induces upregulation and activation of caspases 1, 3, and 8 to produce apoptosis in human erythroid progenitor cells. Blood 1999; 93:3309-16.
24. Jain N, Sudhakar C, Swarup G. Tumor necrosis factor-alpha-induced caspase-1 gene expression. Role of p73. FEBS J 2007; 274:4396-407.
25. Choubey D, Duan X, Dickerson E, et al. Interferon-inducible p200-family proteins as novel sensors of cytoplasmic DNA: role in inflammation and autoimmunity. J Interferon Cytokine Res 2010; 30:371-80.
26. Choubey D. DNA-responsive inflammasomes and their regulators in autoimmunity. Clin Immunol 2012; 142:223-31.
27. Sharma D, Malik A, Guy C, Vogel P, Kanneganti T D. TNF/TNFR axis promotes pyrin inflammasome activation and distinctly modulates pyrin inflammasomopathy. J Clin Invest 2019; 129:150-62.
28. Buddi R, Lin B, Atilano S R, Zorapapel N C, Kenney M C, Brown D J. Evidence of oxidative stress in human corneal diseases. J Histochem Cytochem 2002; 50:341-51.
29. Jurkunas U V, Bitar M S, Funaki T, Azizi B. Evidence of oxidative stress in the pathogenesis of fuchs endothelial corneal dystrophy. Am J Pathol 2010; 177:2278-89.
30. Zhao X, Wang Y, Wang Y, Li S, Chen P. Oxidative stress and premature senescence in corneal endothelium following penetrating keratoplasty in an animal model. BMC Ophthalmol 2016; 16:16.
31. Piippo N, Korhonen E, Hytti M, Kinnunen K, Kaarniranta K, Kauppinen A. Oxidative Stress is the Principal Contributor to Inflammasome Activation in Retinal Pigment Epithelium Cells with Defunct Proteasomes and Autophagy. Cell Physiol Biochem 2018; 49:359-67.
32. Shimizu H, Sakimoto T, Yamagami S. Pro-inflammatory role of NLRP3 inflammasome in experimental sterile corneal inflammation. Sci Rep 2019; 9:9596.
33. Bian F, Xiao Y, Zaheer M, et al. Inhibition of NLRP3 Inflammasome Pathway by Butyrate Improves Corneal Wound Healing in Corneal Alkali Burn. Int J Mol Sci 2017; 18.
34. Landou I, Engler C, Mehrle S, et al. Role of human corneal endothelial cells in T-cell-mediated alloimmune attack in vitro. Invest Ophthalmol Vis Sci 2014; 55:1213-21.
35. Brand F J, 3rd, Forouzandeh M, Kaur H, Travascio F, de Rivero Vaccari J P. Acidification changes affect the inflammasome in human nucleus pulposus cells. J Inflamm (Lond) 2016; 13:29.
36. Mejias N H, Martinez C C, Stephens M E, de Rivero Vaccari J P. Contribution of the inflammasome to inflammaging. J Inflamm (Lond) 2018; 15:23.
37. Man S M, Kanneganti T D. Gasdermin D: the long-awaited executioner of pyroptosis. Cell Res 2015; 25:1183-4.
38. Harijith A, Ebenezer D L, Natarajan V. Reactive oxygen species at the crossroads of inflammasome and inflammation. Front Physiol 2014; 5:352.
39. Juthani V V, Clearfield E, Chuck R S. Non-steroidal anti-inflammatory drugs versus corticosteroids for controlling inflammation after uncomplicated cataract surgery. Cochrane Database Syst Rev 2017; 7:CD010516.
40. Ing J J, Ing H H, Nelson L R, Hodge D O, Bourne W M. Ten-year postoperative results of penetrating keratoplasty. Ophthalmology 1998; 105:1855-65.
41. Yamaguchi T, Higa K, Tsubota K, Shimazaki J. Elevation of preoperative recipient aqueous cytokine levels in eyes with primary graft failure after corneal transplantation. Mol Vis 2018; 24:613-20.
42. Yagi-Yaguchi Y, Yamaguchi T, Higa K, et al. Preoperative Aqueous Cytokine Levels Are Associated With a Rapid Reduction in Endothelial Cells After Penetrating Keratoplasty. Am J Ophthalmol 2017; 181:166-73.
43. Andrei C, Dazzi C, Lotti L, Torrisi M R, Chimini G, Rubartelli A. The secretory route of the leaderless protein interleukin 1beta involves exocytosis of endolysosome-related vesicles. Mol Biol Cell 1999; 10:1463-75.
44. Lopez-Castejon G, Brough D. Understanding the mechanism of IL-1beta secretion. Cytokine Growth Factor Rev 2011; 22:189-95.
45. MacKenzie A, Wilson H L, Kiss-Toth E, Dower S K, North R A, Surprenant A. Rapid secretion of interleukin-1beta by microvesicle shedding. Immunity 2001; 15:825-35.
46. Fink S L, Cookson B T. Caspase-1-dependent pore formation during pyroptosis leads to osmotic lysis of infected host macrophages. Cell Microbiol 2006; 8:1812-25.
47. Kovacs S B, Miao E A. Gasdermins: Effectors of Pyroptosis. Trends Cell Biol 2017; 27:673-84.
48. Liu X, Zhang Z, Ruan J, et al. Inflammasome-activated gasdermin D causes pyroptosis by forming membrane pores. Nature 2016; 535:153-8.
49. He W T, Wan H, Hu L, et al. Gasdermin D is an executor of pyroptosis and required for interleukin-1beta secretion. Cell Res 2015; 25:1285-98.
50. Nemet A Y, Assia E I, Meyerstein D, Meyerstein N, Gedanken A, Topaz M. Protective effect of free-radical scavengers on corneal endothelial damage in phacoemulsification. J Cataract Refract Surg 2007; 33:310-5.
51. Alfonso-Loeches S, Urena-Peralta J R, Morillo-Bargues M J, Oliver-De La Cruz J, Guerri C. Role of mitochondria ROS generation in ethanol-induced NLRP3 inflammasome activation and cell death in astroglial cells. Front Cell Neurosci 2014; 8:216.
52. Zheng Q, Ren Y, Reinach P S, et al. Reactive oxygen species activated NLRP3 inflammasomes prime environment-induced murine dry eye. Exp Eye Res 2014; 125:1-8.
53. Gao J, Liu R T, Cao S, et al. NLRP3 inflammasome: activation and regulation in age-related macular degeneration. Mediators Inflamm 2015; 2015:690243.
54. Gao J, Cui J Z, To E, Cao S, Matsubara J A. Evidence for the activation of pyroptotic and apoptotic pathways in RPE cells associated with NLRP3 inflammasome in the rodent eye. J Neuroinflammation 2018; 15:15.
55. Chi W, Li F, Chen H, et al. Caspase-8 promotes NLRP1/NLRP3 inflammasome activation and IL-1beta production in acute glaucoma. Proc Natl Acad Sci USA 2014; 111:11181-6.

What is claimed:

1. A method of treating or preventing corneal dysfunction in a mammalian subject in need thereof, the method comprising administering to the subject an inflammasome inhibitor in an amount effective to treat or prevent the corneal dysfunction.

2. A method of treating or preventing ocular surface disease in a mammalian subject in need thereof, the method comprising administering to the subject an inflammasome inhibitor in an amount effective to treat or prevent the ocular surface disease or damage.

3. The method of claim 1 or 2, wherein the inflammasome inhibitor is an antisense oligonucleotide or CRISPR Cas9 protein and one or more guide RNA molecules, TALEN or zinc finger nuclease (ZFN) targeting caspase-1, caspase-4, caspase-5, caspase-11, caspase-8, ASC, NLRP1, NLRP2, NLRP3, pyrin, AIM2, NLRP6, NLRP12 or any other protein forming protein-protein interactions with ASC.

4. The method of claim 1 or 2, wherein the inflammasome inhibitor is an anti-caspase-1 antibody, anti-caspase-4 antibody, anti-caspase-5 antibody, anti-caspase-11 antibody, anti-caspase-8 antibody, anti-ASC antibody, anti-NLRP1 antibody, anti-NLRP2 antibody, anti-NLRP3 antibody, anti-pyrin antibody, anti-NLRP6 antibody, anti-NLRP12 antibody or anti-AIM2 antibody.

5. The method of claim 1 or 2, wherein the inflammasome inhibitor is an siRNA that inhibits the expression of caspase-1, caspase-4, caspase-5, caspase-11, caspase-8, ASC, NLRP1, NLRP2, NLRP3, pyrin, AIM2, NLRP6, NLRP12 or any other protein forming protein-protein interactions with ASC.

6. The method of claim 1 or 2, wherein the inflammasome inhibitor is an inhibitor of ASC.

7. The method of claim 6, wherein the inflammasome inhibitor is an antisense oligonucleotide or CRISPR Cas9 protein and one or more guide RNA molecules, TALEN or zinc finger nuclease (ZFN) targeting ASC.

8. The method of claim 6, wherein the inflammasome inhibitor is an anti-ASC antibody.

9. The method of claim 6, wherein the inflammasome inhibitor is an siRNA that inhibits expression of ASC.

10. The method of claim 1 or 2, wherein the inflammasome inhibitor is an inhibitor of caspase-1.

11. The method of claim 10, wherein the inflammasome inhibitor is an antisense oligonucleotide or CRISPR Cas9 protein and one or more guide RNA molecules, TALEN or zinc finger nuclease (ZFN) targeting caspase-1.

12. The method of claim 10, wherein the inflammasome inhibitor is an anti-caspase-1 antibody.

13. The method of claim 10, wherein the inflammasome inhibitor is an siRNA that inhibits expression of caspase-1.

14. The method of any one of claims 1 and 3-13, wherein the corneal dysfunction is corneal endothelial dysfunction, acute or chronic corneal endothelial cell (CEC) loss, bullous keratopathy (PBK), Fuchs' endothelial dystrophy, corneal transplant failure, corneal transplant rejection, corneal inflammation, corneal edema, corneal degeneration, corneal melting, or corneal ectasia.

15. The method of any one of claims 2-13, wherein the ocular surface disease is dry eye syndrome, meibomian gland dysfunction, blepharitis, ocular rosacea allergic conjunctivitis, chemical and thermal ocular burns, Pterygium, Pinguecula, conjunctivochalasis or limbal stem cell deficiency.

16. The method of claim 15, wherein the ocular surface disease is seasonal allergic conjunctivitis (SAC), perennial allergic conjunctivitis (PAC), atopic keratoconjunctivitis (AKC), vernal keratoconjunctivitis (VKC), or giant papillary conjunctivitis (GPC).

17. The method of any one of claims 1-16, wherein administering the inflammasome inhibitor reduces levels of at least one inflammatory cytokine in the cornea or the protein levels of caspase-1, caspase-8, caspase-4, caspase-5, caspase-11, or ASC.

18. The method of any one of claims 1-17, wherein the inflammasome inhibitor is administered topically or by injection to the eye.

19. The method of claim 2, wherein the ocular surface damage is corneal staining.

20. The method of claim 19, further comprising determining an elevated level of caspase-1 in a tear sample from the subject before the administering step.

21. The method of claim 19, wherein the level of caspase-1 in the tear sample is compared to a level of caspase-2 in a control sample.

22. The method of claim 21, wherein the control sample is a tear sample from a healthy subject.

23. The method of any one of claims 20-22, wherein the elevated level of caspase-1 in the tear sample comprises an amount that is at least two standard errors higher than the amount of caspase-1 in the tear sample from the healthy subject.

24. A method of diagnosing ocular surface damage in a mammalian subject in need thereof, the method comprising:
determining a level of caspase-1 in a tear sample from the subject, wherein an elevated level of caspase-1 in the sample compared to a level of caspase-1 control sample identifies the subject as likely to be suffering from ocular surface damage.

25. The method of claim 24, wherein the ocular surface damage is corneal staining.
